SEQUENCE LISTING (96 sequences)

SEQ ID NO: 1 - protein, 404 aa, Pichia pastoris
MAKADGSLLYYNPHNPPRRYYFYMAIFAVSVICVLYGPSQQLSSPKIDYDPLTLRSLDLKTLEAPSQLSPGTVEDNLRRQLEFHFPYRSYEPFPQHIWQTWKVSPSDSSFPKNFKDLGESWLQRSPNYDHFVIPDDAAWELIHHEYERVPEVLEAFHLLPEPILKADFFRYLILFARGGLYADMDTMLLKPIESWLTFNETIGGVKNNAGLVIGIEADPDRPDWHDWYARRIQFCQWAIQSKRGHPALRELIVRVVSTTLRKEKSGYLNMVEGKDRGSDVMDWTGPGIFTDTLFDYMTNVNTTGHSGQGIGAGSAYYNALSLEERDALSARPNGEMLKEKVPGKYAQQVVLWEQFTNLRSPKLIDDILILPITSFSPGIGHSGAGDLNHHLAYIRHTFEGSWKD

SEQ ID NO: 2 - protein, 462 aa, Pichia pastoris
MSTDSNLGYGISISGGSRSTQSLGTSRVTPSRSANHEGKENKAFSMISPKKLINKLSKSSVSSNNTSSSNHDSFVDRKYKIEIENSFSDRSVSEVDLLEDSLDTTEGDSGENLVSTPTQVTLRPKRGNSQDRNENRVLKEKETAVRESQRHTGFFTESMLSPSDGSRQDTSDSPGSISIPTAELSKKNLSDVSKSTSENSHNRKWEARSSLLPENLSSIHLDDSPIEIYEDAEEIIDETVEEPRSSIPLQNEWEMEDTILEGRLVQSASDPVITSNDISKELRKSISTPALTHSDLVDFRKVIPGSSHYHVFTDPKSPFTEDPSQLAYHKIRDRNFDAHYSTDPIRLSSGSSSEGSDEKNLLLGSRKPSDPYRLPYEDEDGYRFWTKTPLNRECPKRVALWLLVGAILAPPVWIMMYVGFLDSSVGRLPPKYRVISGVLALSMIILTAMGIAVGFAYGLNNR

SEQ ID NO: 3 - protein, 652 aa, Pichia pastoris
MFKETSKNLFGSINTFNTVEYVMYMMLLLTAYFLNHLLHSLDNINHLVESDVNYQLLQRVTNKVKLFDEEAVLPFAKNLNRRTERFDPRLPVAAYLRSLQDQYSELPQGTDLNDIPPLEVSFHWDDWLSLGIASTFWDAFDNYNKRQGENAISYEQLQAILVNDLEDFSPYTAHILHSNVEVYKYRTIPQKIVYMSNKGYFELLVTEKEKLSNEGLWSIFHQKQGGLNEFSSLNLIEEVDALDEIYDSKGLPAWDPPFPEELDASDEDLPFNATEELAKVEQIKEPKLEDIFYQEGLQHGIQTLPSDASVYFPVNYVENDPGLQSHHLHFPFFSGMVLPREIHSSVHHMNKAFFLFARQHGYVVWFFYGNLIGWYYNGNNHPWDSDIDAIMPMAEMARMAHHHNNTLIIENPHDGYGTYLLTISPWFTKKTRGGNHIDGRFVDVKRGTYIDLSAISAMHGIYPDWVRDGVKENPKNLALADKNGNWYLTRDILPLRRTIFEGSRSYTVKDIEDTLLRNYGDKVLINTELADHEWHDDWKMWVQKKKYCTYEEFEDYLSAHGGVEYDEDGVLTLEGACGFEEVRQDWIITRESVNLHMKEWEAIQRNESTTEYTAKDLPRYRPDSFKNLLDGVSNHGNGNVGKIEHVKLEHND

SEQ ID NO: 4 - protein, 594 aa, Pichia pastoris
MRIRSNVLLLSTAGALALVWFAVVFSWDDKSIFGIPTPGHAVASAYDSSVTLGTFNDMEVDSYVTNIYDNAPVLGCYDLSYHGLLKVSPKHEILCDMKFIRARVLETEAYAALKDLEHKKLTEEEKIEKHWFTFYGSSVFLPDHDVHYLVRRVVFSGEGKANRPITSILVAQIYDKNWNELNGHFLNVLNPNTGKLQHHAFPQVLPIAVNWDRNSKYRGQEDPRVVLRRGRFGPDPLVMFNTLTQNNKLRRLFTISPFDQYKTVMYRTNAFKMQTTEKNWVPFFLKDDQESVHFVYSFNPLRVLNCSLDNGACDVLFELPHDFGMSSELRGATPMLNLPQAIPMADDKEIWVSFPRTRISDCGCSETMYRPMLMLFVREGTNFFAELLSSSIDFGLEVIPYTGDGLPCSSGQSVLIPNSIDNWEVTGSNGEDILSLTFSEADKSTSVVHIRGLYKYLSELDGYGGPEAEDEHNFQRILSDLHFDGKKTIENFKKVQSCALDAAKAYCKEYGVTRGEEDRLKNKEKERKIEEKRKKEEERKKKEEEKKKKEEEEKKKKEEEEEEEKRLKELKKKLKELQEELEKQKDEVKDTKAK

SEQ ID NO: 5 - protein, 644 aa, Pichia pastoris
MRTRLNFLLLCIASVLSVIWIGVLLTWNDNNLGGISLNGGKDSAYDDLLSLGSFNDMEVDSYVTNIYDNAPVLGCTDLSYHGLLKVTPKHDLACDLEFIRAQILDIDVYSAIKDLEDKALTVKQKVEKHWFTFYGSSVFLPEHDVHYLVRRVIFSAEGKANSPVTSIIVAQIYDKNWNELNGHFLDILNPNTGKVQHNTFPQVLPIATNFVKGKKFRGAEDPRVVLRKGRFGPDPLVMFNSLTQDNKRRRIFTISPFDQFKTVMYDIKDYEMPRYEKNWVPFFLKDNQEAVHFVYSFNPLRVLKCSLDDGSCDIVFEIPKVDSMSSELRGATPMINLPQAIPMAKDKEIWVSFPRTRIANCGCSRTTYRPMLMLFVREGSNFFVELLSTSLDFGLEVLPYSGNGLPCSADHSVLIPNSIDNWEVVDSNGDDILTLSFSEADKSTSVIHIRGLYNYLSELDGYQGPEAEDEHNFQRILSDLHFDNKTTVNNFIKVQSCALDAAKGYCKEYGLTRGEAERRRRVAEERKKKEKEEEEKKKKKEKEEEEKKRIEEEKKKIEEKERKEKEKEEAERKKLQEMKKKLEEITEKLEKGQRNKEIDPKEKQREEEERKERVRKIAEKQRKEAEKKEAEKKANDKKDLKIRQ

SEQ ID NO: 6 - protein, 488 aa, Pichia pastoris
MYHLAPRKKLLIWGGSLGFVLLLLIVASSHQRIRSTILHRTPISTLPVISQEVITADYHPTLLTGFIPTDSDDSDCADFSPSGVIYSTDKLVLHDSLKDIRDSLLKTQYKDLVTLEDEEKMNIDDILKRWYTLSGSSVWIPGMKAHLVVSRVMYLGTNGRSDPLVSFVRVQLFDPDFNELKDIALKFSDKPDGTVIFPYILPVDIPREGSRWLGPEDAKIAVNPETPDDPIVIFNMQNSVNRAMYGFYPFRPENKQVLFSIKDEEPRKKEKNWTPFFVPGSPTTVNFVYDLQKLTILKCSIITGICEKEFVSGDDGQNHGIGIFRGGSNLVPFPTSFTDKDVWVGFPKTHMESCGCSSHIYRPYLMVLVRKGDFYYKAFVSTPLDFGIDVRSWESAESTSCQTAKNVLAVNSISNWDLLDDGLDKDYMTITLSEADVVNSVLRVRGIAKFVDNLTMDDGSTTLSTSNKIDECATTGSKQYCQRYGELH

SEQ ID NO: 7 - protein, 652 aa, Pichia pastoris
MVDLFQWLKFYSMRRLGQVAITLVLLNLFVFLGYKFTPSTVIGSPSWEPAVVPTVFNESYLDSLQFTDINVDSFLSDTNGRISVTCDSLAYKGLVKTSKKKELDCDMAYIRRKIFSSEEYGVLADLEAQDITEEQRIKKHWFTFYGSSVYLPEHEVHYLVRRVLFSKVGRADTPVISLLVAQLYDKDWNELTPHTLEIVNPATGNVTPQTFPQLIHVPIEWSVDDKWKGTEDPRVFLKPSKTGVSEPIVLFNLQSSLCDGKRGMFVTSPFRSDKVNLLDIEDKERPNSEKNWSPFFLDDVEVSKYSTGYVHFVYSFNPLKVIKCSLDTGACRMIYESPEEGRFGSELRGATPMVKLPVHLSLPKGKEVWVAFPRTRLRDCGCSRTTYRPVLTLFVKEGNKFYTELISSSIDFHIDVLSYDAKGESCSGSISVLIPNGIDSWDVSKKQGGKSDILTLTLSEADRNTVVVHVKGLLDYLLVLNGEGPIHDSHSFKNVLSTNHFKSDTTLLNSVKAAECAIFSSRDYCKKYGETRGEPARYAKQMENERKEKEKKEKEAKEKLEAEKAEMEEAVRKAQEAIAQKEREKEEAEQEKKAQQEAKEKEAEEKAAKEKEAKENEAKKKIIVEKLAKEQEEAEKLEAKKKLYQLQEEERS

SEQ ID NO: 8 - protein, 789 aa, Pichia pastoris
MCQIFLPQNVTRCSVSLLTMSKTSPQEVPENTTELKISKGELRPFIVTSPSPQLSKSRSVTSTKEKLILASLFIFAMVIRFHNVAHPDSVVFDEVHFGGFARKYILGTFFMDVHPPLAKLLFAGVGSLGGYDGEFEFKKIGDEFPENVPYVLMRYLPSGMGVGTCIMLYLTLRASGCQPIVCCSDNRSLIIENANVTISRFILLDSPMLFFIASTVYSFKKFQIQEPFTFQWYKTLIATGVSLGLAASSKWVGLFTVAWIGLITIWDLWFIIGDLTVSVKKIFGHFITRAVAFLVVPTLIYLTFFAIHLQVLTKEGDGGAFMSSVFRSTLEGNAVPKQSLANVGLGSLVTIRHLNTRGGYLHSHNHLYEGGSGQQQVTLYPHIDSNNQWIVQDYNATEEPTEFVPLKDGVKIRLNHKLTSRRLHSHNLRPPVTEQDWQNEVSAYGHEGFGGDANDDFVVEIAKDLSTTEEAKENVRAIQTVFRLRHAMTGCYLFSHEVKLPKWAYEQQEVTCATQGIKPLSYWYVETNENPFLDKEVDEIVSYPVPTFFQKVAELHARMWKINKGLTDHHVYESSPDSWPFLLRGISYWSKNHSQIYFIGNAVTWWTVTASIALFSVFLVFSILRWQRGFGFSVDPTVFNFNVQMLHYILGWVLHYLPSFLMARQLFLHHYLPSLYFGILALGHVFEIIHSYVFKNKQVVSYSIFVLFFAVALSFFQRYSPLIYAGRWTKDQCNESKILKWDFDCNTFPSHTSQYEIWASPVQTSTPKEGTHSESTVGEPDVEKLGETV

SEQ ID NO: 9 - protein, 512 aa, Artificial sequence
  Synthesized
EAEAYPKPGATKRGSPNPTRAAAVKAAFQTSWNAYHHFAFPHDDLHPVSNSFDDERNGWGSSAIDGLDTAILMGDADIVNTILQYVPQINFTTTAVANQGISVFETNIRYLGGLLSAYDLLRGPFSSLATNQTLVNSLLRQAQTLANGLKVAFTTPSGVPDPTVFFNPTVRRSGASSNNVAEIGSLVLEWTRLSDLTGNPQYAQLAQKGESYLLNPKGSPEAWPGLIGTFVSTSNGTFQDSSGSWSGLMDSFYEYLIKMYLYDPVAFAHYKDRWVLAADSTIAHLASHPSTRKDLTFLSSYNGQSTSPNSGHLASFAGGNFILGGILLNEQKYIDFGIKLASSYFATYNQTASGIGPEGFAWVDSVTGAGGSPPSSQSGFYSSAGFWVTAPYYILRPETLESLYYAYRVTGDSKWQDLAWEAFSAIEDACRAGSAYSSINDVTQANGGGASDDMESFWFAEALKYAYLIFAEESDVQVQANGGNKFVFNTEAHPFSIRSSSRRGGHLAHDEL

SEQ ID NO: 10 - protein, 445 aa, Artificial sequence
  Synthesized
MSLSLVSYRLRKNPWVNIFLPVLAIFLIYIIFFQRDQSSVSALDGDPASLTREVIRLAQDAEVELERQRGLLQQIGDALSSQRGRVPTAAPPAQPRVPVTPAPAVIPILVIACDRSTVRRCLDKLLHYRPSAELFPIIVSQDCGHEETAQAIASYGSAVTHIRQPDLSSIAVPPDHRKFQGYYKIARHYRWALGQVFRQFRFPAAVVVEDDLEVAPDFFEYFRATYPLLKADPSLWCVSAWNDNGKEQMVDASRPELLYRTDFFPGLGWLLLAELWAELEPKWPKAFWDDWMRRPEQRQGRACIRPEISRTMTFGRKGVSHGQFFDQHLKFIKLNQQFVHFTQLDLSYLQREAYDRDFLARVYGAPQLQVEKVRTNDRKELGEVRVQYTGRDSFKAFAKALGVMDDLKSGVPRAGYRGIVTFQFRGRRVHLAPPLTWEGYDPSWN

SEQ ID NO: 11 - protein, 804 aa, Artificial sequence
  Synthesized
MALFLSKRLLRFTVIAGAVIVLLLTLNSNSRTQQYIPSSISAAFDFTSGSISPEQQVISEENDAKKLEQSALNSEASEDSEAMDEESKALKAAAEKADAPIGGGPAGMRVLVTGGSGYIGSHTCVQLLQNGHDVIILDNLCNSKRSVLPVIERLGGKHPTFVEGDIRNEALMTEILHDHAIDTVIHFAGLKAVGESVQKPLEYYDNNVNGTLRLISAMRAANVKNFIFSSSATVYGDQPKIPYVESFPTGTPQSPYGKSKLMVEQILTDLQKAQPDWSIALLRYFNPVGAHPSGDMGEDPQGIPNNLMPYIAQVAVGRRDSLAIFGNDYPTEDGTGVRDYIHVMDLADGHVVAMEKLANKPGVHIYNLGAGVGNSVLDVVNAFSKACGKPVNYHFAPRREGDLPAYWADASKADRELNWRVTRTLDEMAQDTWHWQSRHPQGYPDGTGGGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 12 - protein, 1101 aa, Artificial sequence
  Synthesized
MLLTKRFSKLFKLTFIVLILCGLFVITNKYMDENTSPAGVEDGPKSSQSNFSQGAGSHLLPSQLSLSVDTADCLFASQSGSHNSDVQMLDVYSLISFDNPDGGVWKQGFDITYESNEWDTEPLQVFVVPHSHNDPGWLKTFNDYFRDKTQYIFNNMVLKLKEDSRRKFIWSEISYLSKWWDIIDIQKKDAVKSLIENGQLEIVTGGWVMPDEATPHYFALIDQLIEGHQWLENNIGVKPRSGWAIDPFGHSPTMAYLLNRAGLSHMLIQRVHYAVKKHFALHKTLEFFWRQNWDLGSVTDILCHMMPFYSYDIPHTCGPDPKICCQFDFKRLPGGRFGCPWGVPPETIHPGNVQSRARMLLDQYRKKSKLFRTKVLLAPLGDDFRYCEYTEWDLQFKNYQQLFDYMNSQSKFKVKIQFGTLSDFFDALDKADETQRDKGQSMFPVLSGDFFTYADRDDHYWSGYFTSRPFYKRMDRIMESHLRAAEILYYFALRQAHKYKINKFLSSSLYTALTEARRNLGLFQHHDAITGTAKDWVVVDYGTRLFHSLMVLEKIIGNSAFLLIGKDKLTYDSYSPDTFLEMDLKQKSQDSLPQKNIIRLSAEPRYLVVYNPLEQDRISLVSVYVSSPTVQVFSASGKPVEVQVSAVWDTANTISETAYEISFRAHIPPLGLKVYKILESASSNSHLADYVLYKNKVEDSGIFTIKNMINTEEGITLENSFVLLRFDQTGLMKQMMTKEDGKHHEVNVQFSWYGTTIKRDKSGAYLFLPDGNAKPYVYTTPPFVRVTHGRIYSEVTCFFDHVTHRVRLYHIQGIEGQSVEVSNIVDIRKVYNREIAMKISSDIKSQNRFYTDLNGYQIQPRMTLSKLPLQANVYPMTTMAYIQDAKHRLTLLSAQSLGVSSLNSGQIEVIMDRRLMQDDNRGLEQGIQDNKITANLFRILLEKRSAVNTEEEKKSVSYPSLLSHITSSLMNHPVIPMANKFSSPTLELQGEFSPLQSSLPCDIHLVNLRTIQSKVGNGHSNEAALILHRKGFDCRFSSKGTGLFCSTTQGKILVQKLLNKFIVESLTPSSLSLMHSPPGTQNISEINLSPMEISTFRIQLR

SEQ ID NO: 13 - protein, 394 aa, Artificial sequence
  Synthesized
MLLTKRFSKLFKLTFIVLILCGLFVITNKYMDENTSPAGSLVYQLNFDQTLRNVDKAGTWAPRELVLVVQVHNRPEYLRLLLDSLRKAQGIDNVLVIFSHDFWSTEINQLIAGVNFCPVLQVFFPFSIQLYPNEFPGSDPRDCPRDLPKNAALKLGCINAEYPDSFGHYREAKFSQTKHHWWWKLHFVWERVKILRDYAGLILFLEEDHYLAPDFYHVFKKMWKLKQQECPECDVLSLGTYSASRSFYGMADKVDVKTWKSTEHNMGLALTRNAYQKLIECTDTFCTYDDYNWDWTLQYLTVSCLPKFWKVLVPQIPRIFHAGDCGMHHKKTCRPSTQSAQIESLLNNNKQYMFPETLTISEKFTVVAISPPRKNGGWGDIRDHELCKSYRRLQ

SEQ ID NO: 14 - DNA, 1539 bp, Artificial sequence
  Synthesized
gaggctgaagcttatccaaagccgggcgccacaaaacgtggatctcccaaccctacgagggcggcagcagtcaaggccgcattccagacgtcgtggaacgcttaccaccattttgcctttccccatgacgacctccacccggtcagcaacagctttgatgatgagagaaacggctggggctcgtcggcaatcgatggcttggacacggctatcctcatgggggatgccgacattgtgaacacgatccttcagtatgtaccgcagatcaacttcaccacgactgcggttgccaaccaaggcatctccgtgttcgagaccaacattcggtacctcggtggcctgctttctgcctatgacctgttgcgaggtcctttcagctccttggcgacaaaccagaccctggtaaacagccttctgaggcaggctcaaacactggccaacggcctcaaggttgcgttcaccactcccagcggtgtcccggaccctaccgtcttcttcaaccctaccgtccggagaagtggtgcatctagcaacaacgtcgctgaaattggaagcctggtgctcgaatggacacggttgagcgacctgacgggaaacccgcagtatgcccagcttgcgcagaagggcgagtcgtatctcctgaatccaaagggaagcccggaggcatggcctggcctgattggaacgtttgtcagcacgagcaacggtacctttcaggatagcagcggcagctggtccggcctcatggacagcttctacgagtacctgatcaagatgtacctgtacgacccggttgcgtttgcacactacaaggatcgctgggtccttgctgccgactcgaccattgcgcatctcgcctctcacccgtcgacgcgcaaggacttgacctttttgtcttcgtacaacggacagtctacgtcgccaaactcaggacatttggccagttttgccggtggcaacttcatcttgggaggcattctcctgaacgagcaaaagtacattgactttggaatcaagcttgccagctcgtactttgccacgtacaaccagacggcttctggaatcggccccgaaggcttcgcgtgggtggacagcgtgacgggcgccggcggctcgccgccctcgtcccagtccgggttctactcgtcggcaggattctgggtgacggcaccgtattacatcctgcggccggagacgctggagagcttgtactacgcataccgcgtcacgggcgactccaagtggcaggacctggcgtgggaagcgttcagtgccattgaggacgcatgccgcgccggcagcgcgtactcgtccatcaacgacgtgacgcaggccaacggcgggggtgcctctgacgatatggagagcttctggtttgccgaggcgctcaagtatgcgtacctgatctttgcggaggagtcggatgtgcaggtgcaggccaacggcgggaacaaatttgtctttaacacggaggcgcacccctttagcatccgttcatcatcacgacggggcggccaccttgctcacgacgagttgtaa

SEQ ID NO: 15 - DNA, 1338 bp, Artificial sequence
  Synthesized
atgtcactttctcttgtatcgtaccgcctaagaaagaacccgtgggttaacatttttctacctgttttggccatatttctaatatatataatttttttccagagagatcaatcttcagtcagcgctctcgatggcgaccccgccagcctcacccgggaagtgattcgcctggcccaagacgccgaggtggagctggagcggcagcgtgggctgctgcagcagatcggggatgccctgtcgagccagcgggggagggtgcccaccgcggcccctcccgcccagccgcgtgtgcctgtgacccccgcgccggcggtgattcccatcctggtcatcgcctgtgaccgcagcactgttcggcgctgcctggacaagctgctgcattatcggccctcggctgagctcttccccatcatcgttagccaggactgcgggcacgaggagacggcccaggccatcgcctcctacggcagcgcggtcacgcacatccggcagcccgacctgagcagcattgcggtgccgccggaccaccgcaagttccagggctactacaagatcgcgcgccactaccgctgggcgctgggccaggtcttccggcagtttcgcttccccgcggccgtggtggtggaggatgacctggaggtggccccggacttcttcgagtactttcgggccacctatccgctgctgaaggccgacccctccctgtggtgcgtctcggcctggaatgacaacggcaaggagcagatggtggacgccagcaggcctgagctgctctaccgcaccgactttttccctggcctgggctggctgctgttggccgagctctgggctgagctggagcccaagtggccaaaggccttctgggacgactggatgcggcggccggagcagcggcaggggcgggcctgcatacgccctgagatctcaagaacgatgacctttggccgcaagggtgtgagccacgggcagttctttgaccagcacctcaagtttatcaagctgaaccagcagtttgtgcacttcacccagctggacctgtcttacctgcagcgggaggcctatgaccgagatttcctcgcccgcgtctacggtgctccccagctgcaggtggagaaagtgaggaccaatgaccggaaggagctgggggaggtgcgggtgcagtatacgggcagggacagcttcaaggctttcgccaaggctctgggtgtcatggatgaccttaagtcgggggttccgagagctggctaccggggtattgtcaccttccagttccggggccgccgtgtccacctggcgcccccactgacgtgggagggctatgatcctagctggaattag

SEQ ID NO: 16 - DNA, 2397 bp, Artificial sequence
  Synthesized
atggccctctttctcagtaagagactgttgagatttaccgtcattgcaggtgcggttattgttctcctcctaacattgaattccaacagtagaactcagcaatatattccgagttccatctccgctgcatttgattttacctcaggatctatatcccctgaacaacaagtcatctctgaggaaaatgatgctaaaaaattagagcaaagtgctctgaattcagaggcaagcgaagactccgaagccatggatgaagaatccaaggctctgaaagctgccgctgaaaaggcagatgccccgatcatgagagttctggttaccggtggtagcggttacattggaagtcatacctgtgtgcaattactgcaaaacggtcatgatgtcatcattcttgataacctctgtaacagtaagcgcagcgtactgcctgttatcgagcgtttaggcggcaaacatccaacgtttgttgaaggcgatattcgtaacgaagcgttgatgaccgagatcctgcacgatcacgctatcgacaccgtgatccacttcgccgggctgaaagccgtgggcgaatcggtacaaaaaccgctggaatattacgacaacaatgtcaacggcactctgcgcctgattagcgccatgcgcgccgctaacgtcaaaaactttatttttagctcctccgccaccgtttatggcgatcagcccaaaattccatacgttgaaagcttcccgaccggcacaccgcaaagcccttacggcaaaagcaagctgatggtggaacagatcctcaccgatctgcaaaaagcccagccggactggagcattgccctgctgcgctacttcaacccggttggcgcgcatccgtcgggcgatatgggcgaagatccgcaaggcattccgaataacctgatgccatacatcgcccaggttgctgtaggccgtcgcgactcgctggcgatttttggtaacgattatccgaccgaagatggtactggcgtacgcgattacatccacgtaatggatctggcggacggtcacgtcgtggcgatggaaaaactggcgaacaagccaggcgtacacatctacaacctcggcgctggcgtaggcaacagcgtgctggacgtggttaatgccttcagcaaagcctgcggcaaaccggttaattatcattttgcaccgcgtcgcgagggcgaccttccggcctactgggcggacgccagcaaagccgaccgtgaactgaactggcgcgtaacgcgcacactcgatgaaatggcgcaggacacctggcactggcagtcacgccatccacagggatatcccgatggtaccggtggtggacgtgacctttctcgtctgccacaactggttggagtttctactccactgcaaggtggatctaactctgctgctgcaattggtcaatcatctggtgagcttcgtactggaggtgctcgtccccctccaccacttggtgcttcttcccagccccgtccaggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggttggggaggagaagatgacgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctaa

SEQ ID NO: 17 - DNA, 3306 bp, Artificial sequence
  Synthesized
atgctgcttaccaaaaggttttcaaagctgttcaagctgacgttcatagttttgatattgtgcgggctgttcgtcattacaaacaaatacatggatgagaacacgtcgcctgcaggcgtggaggatggtccgaaaagttcacaaagcaatttcagccaaggtgctggctcacatcttctgccctcacaattatccctctcagttgacactgcagactgtctgtttgcttcacaaagtggaagtcacaattcagatgtgcagatgttggatgtttacagtctaatttcttttgacaatccagatggtggagtttggaagcaaggatttgacattacttatgaatctaatgaatgggacactgaaccccttcaagtctttgtggtgcctcattcccataacgacccaggttggttgaagactttcaatgactactttagagacaagactcagtatatttttaataacatggtcctaaagctgaaagaagactcacggaggaagtttatttggtctgagatctcttacctttcaaagtggtgggatattatagatattcagaagaaggatgctgttaaaagtttaatagaaaatggtcagcttgaaattgtgacaggtggctgggttatgcctgatgaagctactccacattattttgccttaattgatcaactaattgaaggacatcagtggctggaaaataatataggagtgaaacctcggtccggctgggctattgatccctttggacactcaccaacaatggcttatcttctaaaccgtgctggactttctcacatgcttatccagagagttcattatgcagttaaaaaacactttgcactgcataaaacattggagtttttttggagacagaattgggatctgggatctgtcacagatattttatgccacatgatgcccttctacagctatgacatccctcacacttgtggacctgatcctaaaatatgctgccagtttgattttaaacgtcttcctggaggcagatttggttgtccctggggagtccccccagaaacaatacatcctggaaatgtccaaagcagggctcggatgctactagatcagtaccgaaagaagtcaaagctttttcgaaccaaagttctcctggctccactaggagatgatttccgctactgtgaatacacggaatgggatttacagtttaagaattatcagcagctttttgattatatgaattctcagtccaagtttaaagttaagatacagtttggaactttatcagatttttttgatgcgctggataaagcagatgaaactcagagagacaagggccaatcgatgttccctgttttaagtggagattttttcacttatgccgatcgagatgatcattactggagtggctattttacatccagacccttttacaaacgaatggacagaatcatggaatctcatttaagggctgctgaaattctttactatttcgccctgagacaagctcacaaatacaagataaataaatttctctcatcatcactttacacggcactgacagaagccagaaggaatttgggactgtttcaacatcatgatgctatcacaggaactgcaaaagactgggtggttgtggattatggtaccagactttttcattcgttaatggttttggagaagataattggaaattctgcatttcttcttattgggaaggacaaactcacatacgactcttactctcctgataccttcctggagatggatttgaaacaaaaatcacaagattctctgccacaaaaaaatataataaggctgagtgcggagccaaggtaccttgtggtctataatcctttagaacaagaccgaatctcgttggtctcagtctatgtgagttccccgacagtgcaagtgttctctgcttcaggaaaacctgtggaagttcaagtcagcgcagtttgggatacagcaaatactatttcagaaacagcctatgagatctcttttcgagcacatataccgccattgggactgaaagtgtataagattttggaatcagcaagttcaaattcacatttagctgattatgtcttgtataagaataaagtagaagatagcggaattttcaccataaagaatatgataaatactgaagaaggtataacactagagaactcctttgttttacttcggtttgatcaaactggacttatgaagcaaatgatgactaaagaagatggtaaacaccatgaagtaaatgtgcaattttcatggtatggaaccacaattaaaagagacaaaagtggtgcctacctcttcttacctgatggtaatgccaagccttatgtttacacaacaccgccctttgtcagagtgacacatggaaggatttattcggaagtgacttgcttttttgaccatgttactcatagagtccgactataccacatacagggaatagaaggacagtctgtggaagtttccaatattgtggacatccgaaaagtatataaccgtgagattgcaatgaaaatttcttctgatataaaaagccaaaatagattttatactgacctaaatgggtaccagattcaacctagaatgacactgagcaaattgcctcttcaagcaaatgtctatcccatgaccacaatggcctatatccaggatgccaaacatcgtttgacactgctctctgctcagtcattaggggtttcgagtttgaatagtggtcagattgaagttatcatggatcgaagactcatgcaagatgataatcgtggccttgagcaaggtatccaggataacaagattacagctaatctatttcgaatactactagaaaaaagaagtgctgttaatacggaagaagaaaagaagtcggtcagttatccttctctccttagccacataacttcttctctcatgaatcatccagtcattccaatggcaaataagttctcctcacctacccttgagctgcaaggtgaattctctccattacagtcatctttgccttgtgacattcatctggttaatttgagaacaatacagtcaaaggtgggcaatgggcactccaatgaggcagccttgatcctccacagaaaagggtttgattgtcggttctctagcaaaggcacagggctgttttgttctactactcagggaaagatattggtacagaaacttttaaacaagtttattgtcgaaagtctcacaccttcatcactatccttgatgcattcacctcccggcactcagaatataagtgagatcaacttgagtccaatggaaatcagcacattccgaatccagttgaggtga

SEQ ID NO: 18 - DNA, 1188 bp, Artificial sequence
  Synthesized
atgctgcttaccaaaaggttttcaaagctgttcaagctgacgttcatagttttgatattgtgcgggctgttcgtcattacaaacaaatacatggatgagaacacgtcgcctgcaggctccctggtgtaccagctgaactttgatcagaccctgaggaatgtagataaggctggcacctgggccccccgggagctggtgctggtggtccaggtgcataaccggcccgaatacctcagactgctgctggactcacttcgaaaagcccagggaattgacaacgtcctcgtcatctttagccatgacttctggtcgaccgagatcaatcagctgatcgccggggtgaatttctgtccggttctgcaggtgttctttcctttcagcattcagttgtaccctaacgagtttccaggtagtgaccctagagattgtcccagagacctgccgaagaatgccgctttgaaattggggtgcatcaatgctgagtatcccgactccttcggccattatagagaggccaaattctcccagaccaaacatcactggtggtggaagctgcattttgtgtgggaaagagtgaaaattcttcgagattatgctggccttatacttttcctagaagaggatcactacttagccccagacttttaccatgtcttcaaaaagatgtggaaactgaagcagcaagagtgccctgaatgtgatgttctctccctggggacctatagtgccagtcgcagtttctatggcatggctgacaaggtagatgtgaaaacttggaaatccacagagcacaatatgggtctagccttgacccggaatgcctatcagaagctgatcgagtgcacagacactttctgtacttatgatgattataactgggactggactcttcaatacttgactgtatcttgtcttccaaaattctggaaagtgctggttcctcaaattcctaggatctttcatgctggagactgtggtatgcatcacaagaaaacctgtagaccatccactcagagtgcccaaattgagtcactcttaaataataacaaacaatacatgtttccagaaactctaactatcagtgaaaagtttactgtggtagccatttccccacctagaaaaaatggagggtggggagatattagggaccatgaactctgtaaaagttatagaagactgcagtgataa

SEQ ID NO: 19 - DNA, 4921 bp, Artificial sequence
  Synthesized
ggcatacactattatcttatctatattagtcgtcgccgttgcttttggatcctcgtgtatctctggagcattattcactgtggaagataattataatgtttcattggaagttgccattttgacagtttcattgatggtcttgggtttctccttgggtccattgttgtggtctcctttatctgagcagattggaaggagatgggtttattttatatccttgggtctctacacaatttttaacattccttgcgctctatcccctaatatcggtggtctcttagtttgtcgatttttgtgtggtgtttttagttccagcgcactttgtctggttggtggttctatagctgacatgcatccttctgaaacaagaggtaaagcaatcgcctattttgcagcagctccttatggtggaccagttattggacctttagtatgtggttggatcggtgttaaaaccaacagaatggatcttatcttttgggtaaatatgggatttgcaggatttatgtggttactagttgcctgcattccagaaacctatcaaccagtaattttaaagaaccgagcaaagaaattaagaatggagttgaacaatcctaacatcatgacagagcaagaagctaatccactaactttcaaggaattagtagttacctgcctttataggcctcttatgtttgttttcactgagcctgttttggacatgatgtgtgtttacgtttgtcttatttactcattgctttatgcatttttctttgcatacccagttatatttaatgagctttatggctatgaagatgatttcatcggcctgatgttgattccaatattgataggagcctttttggccttagttacaactccaattttggaatccatgtacgtgaaaatgtgtcaacgaagaaaaccaactcctgaagacagattggtaggagccatgattgggtctcctttccctgcaattgccctatttattttgggagcaacgtcctacaagcatatcatttgggtcggtccagcatcttccggtatcgccttcggttatggaatggtactaatttactactctttgaataattacatcatcgacacctacgccaagtatgcagctagtgctctggcaacaaaggttttcctgaggagtgctggaggtgctgctttcccactatttactacacagatgtaccataaactagggctacagtgggccagttggttgttggcattcatttcattagcaatgattctcatcccattcgttttctacatttatggtgctcgtttgagggccaaaatgtgtaaagagaactacagtgagatgtgatgcattaagaacaatcattcattaatccttttcagcatatattatttctaattaattcatacttaataacgaaaatatggtacctgccctcacggtggttacggtctaggaacggaacgtatcttagcatggttgtgcgacagattcactgtgaaagactgttcattatacccacgtttcactgggagatgtaagccttaggtgttttaccctgattagataatacaataaccaacagaaatacgagaatctagactaatttcgatgattcatttttctttttaccgcgctgcctcttttggcaattctttcacctatattctaccttctctttccttttgttctaaacttattaccagctatctatgtcgaatcaagaagaaagacttaaactgtggggtggcaggtttactggggctactgaccccttgatggatttgtataacgcttccttaccttacgacaagaaaatgtacaaggtggatttagaaggaacaaaagtttacactgagggcctggagaaaattaatttgctaactaaagacgaactaagtgagattcatcgtggtctcaaattgattgaagcagagtgggcagaagggaagtttgttgagaagccaggggatgaggatattcacactgctaatgaacgtcgcttgggtgagttgattggtcgtggaatctctggtaaggttcataccggaaggtctagaaatgatcaagttgccactgatatgcggttgtatgtcagagacaatctaactcagttggctgactatctgaagcagttcattcaagtaatcatcaagagagctgaacaggaaatagacgtcttgatgcccggttatactcacttgcaaagagctcaaccaatcagatggtctcactggttgagcatgtatgctacctatttcactgaagattatgagagactgaatcaaatcgttaaaaggttgaacaaatccccattgggagctggagctttggctggtcatccttatggaattgatcgtgaatacattgctgagagattagggtttgattctgttattggtaattctttggccgctgtttcagacagagattttgtagtcgaaaccatgttctggtcttcgttgtttatgaatcatatttctcgattctcagaagatttgatcatttactccactggagagtttggatttatcaagttggcagatgcttattctactggatcttctctgatgcctcaaaaaaaaaacccagactctttggagttattgaggggtaaatctggtagatgttttggggccttggctggtttcctcatgtctattaagtccattccgtcaacctataacaaagatatgcaagaggataaggagcctttatttgatactctaatcactgtagagcactcgattttgatagcatccggtgtagtttctaccttgaacattgatgccgaacgaatgaagaatgctctaactatggatatgctggctacagatcttgccgactatttagttagaaggggagttccattcagagaaactcaccacatttctggtgaatgtgtcagacaagccgaggagttgaacctttctggtattgatcagttgtccctcgaacaattgaaatccattgactcccgttttgaggctgatgtggcttcaacgtttgactttgaagccagtgttgaaaaaagaactgccaccggaggaacttctaagactgctgttttaaagcaattggatgcactgaatgaaaagctagagtcttgaaggttttatactgagtttgttaatgatacaataaactgttatagtacatacaattgaaactctcttatctatactgggggaccttctcgcagaatggtataaatatctactaactgactgtcgtacggcctaggggtctcttcttcgattatttgcaggtcggaacatccttcgtctgatgcggatctcctgagacaaagttcacgggtatctagtattctatcagcataaatggaggacctttctaaactaaactttgaatcgtctccagcagcatcctcgcataatccttttgtcatttcctctatgtctattgtcactgtggttggcgcatcaagagtcgtccttctgtaaaccggtacagaattcctaccactagaagcttgaaatggggagggtttcagctttgtatcccgatactgtgctttaaaaagggagtccaaactgaaatctttttcggaatcattggatgatacctctgtattagatctcctatgtatcggtttcctcgggtagatagaacttcactcatcaacattatgatctttgtcgaaaagtatcaattgaaacattgccgctctggctctttccttggtgtccgtgttgtcgctttcaaaactcaatttcttgataacatcataaaatccatctttaattagcttcaacgctcttgatctaggtgctcgcatcttcttgaaatgttcatcggaagttagctcattcaagtacccaacatttatttcttcttcaatagtttccatatccatttcaacatctgaatcttccagatctgaagatgtatcgtccttccatgttaagttggtaactatccaaatacatgatatcatcagatctttatggaaagcggcccattcggaggagaccccttctatttcttgtactaaaggagtctccaataacatataaatgaagtcgagcaattcttgattacaaataatcattgatctgttatcttcattagaggccgcaaaatggaccaggatataagtgatagcaagaataacctcataagtttctgattcctttcttttactaatgtcatcctcctttaatgtggatgataaactcttcaaattttttaatagaaaattcaaaaaatctttatcatcgtgagcttttgctgtcgggtcggaacagaatgattgaatgattttgttcgaatagttaagaggaccacaggacaagtttcggataatattgaatgctttttcttgaatttgcagatttgaagaataacaaagttcaaaaattcttgataaaggaactttgtccaaaaataattttttatcaatgatatcatccccgtaaaggtaatttctaagaattgataaggcattgttctttaagaattcaaactcgttctcttccgaaacaaaataagacaataccttcaggaagtcttcattgaaaacattttttttcaaagaactatattccaccacacaattggaaatgattcccaaaactagcgccttgagtgtaatttcgtcatccaataaggacgcacaagattcattgtctcttgaaaccaaaggcagtttcaccagatccgtcaacgagtttacaatgtttaaatctactaggtaggttcgtagcaatggcgctgatcgtgacagtgagcggaccaagtacagagcagacgtcgtgattatgtttgaaagcttcaatatagtaatgacatgatccaactgagcagcgtctccgtgaaactctttgtaaatattaatatgacgagagacaagatccaccacacattctgaaacaatgtcatgcttgataatttcatctctgtattcctcgttgttggacgtta

SEQ ID NO: 20 - DNA, 2023 bp, Artificial sequence
  Synthesized
ggtaccgcagtttaatcatagcccactgctaagccagaattctaatatgtaactacgtacctttccttttaataaatgatctgtattttccacctagtagcagatcaaattgttcaactttaagtctttggtccctcaagcgagagaacttgcgatgacactcaggagtgccataaaagccagaacctcaaaaggactgatcggagctgttattatagcctcaataatatttttcaccacagtaaccttctacgatgaaagcaaaattgtcggcataataagagtttctgatacttatacaggccatagcgctgtatcttcaactttcaatgcttcttccgttgttagtgacaacaagatcaacggatatggacttcctttgattgacacggaatcaaatagccgttatgaggatccagacgatatttccattgaaaacgaattgcgctatagaattgcccaatctaccaaagaggaagaaaacatgtggaaactcgataccactctcacggaagcaagcttgaaaatccccaacatacagtcgtttgagctgcagccgttcaaagaaagacttgataattcactttacaattctaagaacataggaaacttttacttctatgacccaaggcttacattctcagtttacttgaagtatatcaaggataaattggcctctggaagcacaacaaatcttacaatacccttcaactgggcacattttagagatttatcgtcactgaatccttatttggacataaaacaagaagataaggtcgcatgtgattacttttatgaatcaagtaataaagacaaacgaaaacccacgggtaactgtattgagtttaaagatgttcgtgatgagcacctgatacagtatgggatttcatcaaaagaccatctacctggtccttttattttaaagtcacttggaattcccatgcagcatacagccaagcgactggaatcaaatctttatctattaaccggtgcgccagttccacttgcggccgcactttactttcttggtattggaattcattgatgttcccttgggattatgatattgatgtgcaaatgccaatcaagagtttgaacaatctatgtgctaacttcaaccaatcattaataattgaggatcttactgaaggatattcttcttttttcttggattgcggatcaagtatcacgcatagaacaaaaggcaaaggattaaacttcattgatgcaagattcataaatgttgaaacaggcctttatatcgatatcactggattaagtaccagtcagtcagctcgaccgccaaggtttagtaacgcttcgaagaaagatcctatttacaattgcaggaataatcatttctactctcataacaatatagcacctctcaaatacacgttgatggagggggttcccagtttcattcctcaacagtatgaagaaatattgagagaggagtatacaactggtttgacttcgaaacactacaacggcaacttttttatgactcaattgaatttgtggcttgaaagagatccaatgctagcacttgtgccttcatccaaatacgaaattgaaggtggaggggtggaccataacaagattatcaagtctattcttgaactttccaacatcaaaaaattggaattgttggatgataatcccgatatattagaggaggtgatcaggacatacgaactgacttccattcaccataaagagatgcagtatctttccagtgtcaaaccagatggggacaggtccatgcagtcaaatgacataaccagttcttaccaggagtttctagcaagtctgaagaaattccagcctttacgcaaagatttgttccaatttgagcggatagacctttctaagcatagaaaacagtgagcagccgttttgcctaaaatgttccagaaactataggataaatatatacagtaatgaattaggtgatgttagcatttagtccccaaaaatacctcgaatctccagctccatagcgcaaaatctcggatctaga

SEQ ID NO: 21 - DNA, 32 bp, Artificial sequence
  Synthesized
agtggtaccgcagtttaatcatagcccactgc

SEQ ID NO: 22 - DNA, 53 bp, Artificial sequence
  Synthesized
attccaataccaagaaagtaaagtgcggccgcaagtggaactggcgcaccggt

SEQ ID NO: 23 - DNA, 53 bp, Artificial sequence
  Synthesized
accggtgcgccagttccacttgcggccgcactttactttcttggtattggaat

SEQ ID NO: 24 - DNA, 32 bp, Artificial sequence
  Synthesized
tgttctagatccgagattttgcgctatggagc

SEQ ID NO: 25 - DNA, 25 bp, Artificial sequence
  Synthesized
gcagtttaatcatagcccactgcta

SEQ ID NO: 26 - DNA, 26 bp, Artificial sequence
  Synthesized
agcgtcgatttttgtgatgctcgtca

SEQ ID NO: 27 - DNA, 21 bp, Artificial sequence
  Synthesized
gggaaagaaaaccttcaattt

SEQ ID NO: 28 - DNA, 21 bp, Artificial sequence
  Synthesized
tacaagccagtttcgcaataa

SEQ ID NO: 29 - DNA, 31 bp, Artificial sequence
  Synthesized
agtaggcctttcaacgagtgaccaatgtaga

SEQ ID NO: 30 - DNA, 54 bp, Artificial sequence
  Synthesized
tatctccatagtttctaagcagggcggccgcaatatgtgcggtgtagggagaaa

SEQ ID NO: 31 - DNA, 54 bp, Artificial sequence
  Synthesized
tttctccctacaccgcacatattgcggccgccctgcttagaaactatggagata

SEQ ID NO: 32 - DNA, 30 bp, Artificial sequence
  Synthesized
tgtactagttgaagacgtcccctttgaaca

SEQ ID NO: 33 - DNA, 24 bp, Artificial sequence
  Synthesized
tagtccaagtacgaaacgacacta

SEQ ID NO: 34 - DNA, 22 bp, Artificial sequence
  Synthesized
aaaactatccaatgagggtctc

SEQ ID NO: 35 - DNA, 22 bp, Artificial sequence
  Synthesized
tcttcaatgtctttaacggtgt

SEQ ID NO: 36 - DNA, 34 bp, Artificial sequence
  Synthesized
acttggtaccacacgactcaacttcctgctgctc

SEQ ID NO: 37 - DNA, 35 bp, Artificial sequence
  Synthesized
actgcggccgccacgaaacttcttacctttgacaa

SEQ ID NO: 38 - DNA, 56 bp, Artificial sequence
  Synthesized
ttgtcaaaggtaagaagtttcgtggcggccgctatcttgacattgtcattcagtga

SEQ ID NO: 39 - DNA, 28 bp, Artificial sequence
  Synthesized
caatctagagcctccttcttttccgcct

SEQ ID NO: 40 - DNA, 24 bp, Artificial sequence
  Synthesized
ttttcctcaagccttcaaagacag

SEQ ID NO: 41 - DNA, 18 bp, Artificial sequence
  Synthesized
gggcagaagatcctagag

SEQ ID NO: 42 - DNA, 24 bp, Artificial sequence
  Synthesized
tcgtctccattgctatctacgact

SEQ ID NO: 43 - DNA, 30 bp, Artificial sequence
  Synthesized
tcaacgcgttggctctggatcgttctaata

SEQ ID NO: 44 - DNA, 51 bp, Artificial sequence
  Synthesized
ttctccgttctcctttctccgtgcggccgccagcagcaaggaagataccaa

SEQ ID NO: 45 - DNA, 51 bp, Artificial sequence
  Synthesized
ttggtatcttccttgctgctggcggccgcacggagaaaggagaacggagaa

SEQ ID NO: 46 - DNA, 30 bp, Artificial sequence
  Synthesized
tcaacgcgttggctggaggtgacagaggaa

SEQ ID NO: 47 - DNA, 32 bp, Artificial sequence
  Synthesized
tcaacgcgttagtagtgccgtgccaagtagcg

SEQ ID NO: 48 - DNA, 54 bp, Artificial sequence
  Synthesized
tcctactttgcttatcatctgccgcggccgcggtcaggccctcttatggttgtg

SEQ ID NO: 49 - DNA, 54 bp, Artificial sequence
  Synthesized
cacaaccataagagggcctgaccgcggccgcggcagatgataagcaaagtagga

SEQ ID NO: 50 - DNA, 32 bp, Artificial sequence
  Synthesized
tcaacgcgtcataggtaatggcacagggatag

SEQ ID NO: 51 - DNA, 31 bp, Artificial sequence
  Synthesized
tcaacgcgtgcagcgtttacgaatagtgtcc

SEQ ID NO: 52 - DNA, 52 bp, Artificial sequence
  Synthesized
gcatagggctgaagcatactgtgcggccgcaatgatatgtacgttcccaaga

SEQ ID NO: 53 - DNA, 52 bp, Artificial sequence
  Synthesized
tcttgggaacgtacatatcattgcggccgcacagtatgcttcagccctatgc

SEQ ID NO: 54 - DNA, 33 bp, Artificial sequence
  Synthesized
tcaacgcgtgaggtggacaagagttcaacaaag

SEQ ID NO: 55 - DNA, 24 bp, Artificial sequence
  Synthesized
gttctggtatgcgttctattcttc

SEQ ID NO: 56 - DNA, 22 bp, Artificial sequence
  Synthesized
tatttgccttcttcaccgttat

SEQ ID NO: 57 - DNA, 22 bp, Artificial sequence
  Synthesized
tccgttgagggtgctaatggta

SEQ ID NO: 58 - DNA, 21 bp, Artificial sequence
  Synthesized
tagtctggtttgcggtagtgt

SEQ ID NO: 59 - DNA, 20 bp, Artificial sequence
  Synthesized
agattgagcataggagtggc

SEQ ID NO: 60 - DNA, 20 bp, Artificial sequence
  Synthesized
aaacggagtccagttcttct

SEQ ID NO: 61 - DNA, 20 bp, Artificial sequence
  Synthesized
caactttgcctgtcatttcc

SEQ ID NO: 62 - DNA, 20 bp, Artificial sequence
  Synthesized
cgcttcagttcacggacata

SEQ ID NO: 63 - DNA, 20 bp, Artificial sequence
  Synthesized
gcaacccagacctccttacc

SEQ ID NO: 64 - DNA, 47 bp, Artificial sequence
  Synthesized
tctctcgagaaaagagaggctgaagcttatccaaagccgggcgccac

SEQ ID NO: 65 - DNA, 50 bp, Artificial sequence
  Synthesized
agggaattcttacaactcgtcgtgagcaaggtggccgccccgtcgtgatg

SEQ ID NO: 66 - DNA, 22 bp, Artificial sequence
  Synthesized
gaacacgatccttcagtatgta

SEQ ID NO: 67 - DNA, 22 bp, Artificial sequence
  Synthesized
tgatgatgaacggatgctaaag

SEQ ID NO: 68 - DNA, 28 bp, Artificial sequence
  Synthesized
tcagtcagcgctctcgatggcgaccccg

SEQ ID NO: 69 - DNA, 36 bp, Artificial sequence
  Synthesized
gcgaattcttagtgctaattccagctaggatcatag

SEQ ID NO: 70 - DNA, 151 bp, Artificial sequence
  Synthesized
tataatattatgtcactttctcttgtatcgtaccgcctaagaaagaacccgtgggttaacatttttctacctgttttggccatatttctaatatatataatttttttccagagagatcaatcttcagtcagcgctctcgatggcgaccccg

SEQ ID NO: 71 - DNA, 21 bp, Artificial sequence
  Synthesized
tggacaagctgctgcattatc

SEQ ID NO: 72 - DNA, 21 bp, Artificial sequence
  Synthesized
cggaactggaaggtgacaata

SEQ ID NO: 73 - DNA, 25 bp, Artificial sequence
  Synthesized
atgagagttctggttaccggtggta

SEQ ID NO: 74 - DNA, 31 bp, Artificial sequence
  Synthesized
agggtaccatcgggatatccctgtggatggc

SEQ ID NO: 75 - DNA, 38 bp, Artificial sequence
  Synthesized
atggtaccggtggtggacgtgacctttctcgtctgcca

SEQ ID NO: 76 - DNA, 40 bp, Artificial sequence
  Synthesized
gcatttaaatttagctcggtgtcccgatgtccactgtgat

SEQ ID NO: 77 - DNA, 36 bp, Artificial sequence
  Synthesized
ataatattaaacgatggccctctttctcagtaagag

SEQ ID NO: 78 - DNA, 70 bp, Artificial sequence
  Synthesized
caccggtaaccagaactctcatgatcggggcatctgccttttcagcggcagctttcagagccttggattc

SEQ ID NO: 79 - DNA, 25 bp, Artificial sequence
  Synthesized
tgataacctctgtaacagtaagcgc

SEQ ID NO: 80 - DNA, 25 bp, Artificial sequence
  Synthesized
ggagcttagcacgattgaatatagt

SEQ ID NO: 81 - DNA, 46 bp, Artificial sequence
  Synthesized
ataatattaaaccatgctgcttaccaaaaggttttcaaagctgttc

SEQ ID NO: 82 - DNA, 52 bp, Artificial sequence
  Synthesized
gctatttaaatctattacctcaactggattcggaatgtgctgatttccattg

SEQ ID NO: 83 - DNA, 22 bp, Artificial sequence
  Synthesized
cagatggatgagcatagagtta

SEQ ID NO: 84 - DNA, 22 bp, Artificial sequence
  Synthesized
gacaagaggataatgaagagac

SEQ ID NO: 85 - DNA, 46 bp, Artificial sequence; identical to SEQ ID NO: 81
  Synthesized

SEQ ID NO: 86 - DNA, 40 bp, Artificial sequence
  Synthesized
gctatttaaatttatcactgcagtcttctataacttttac

SEQ ID NO: 87 - DNA, 22 bp, Artificial sequence
  Synthesized
atcaacagtctgatctctagtg

SEQ ID NO: 88 - DNA, 22 bp, Artificial sequence
  Synthesized
agttcatggtccctaatatctc

SEQ ID NO: 89 - DNA, 33 bp, Artificial sequence
  Synthesized
tctacgcgtccttagacatgactgttcctcagt

SEQ ID NO: 90 - DNA, 30 bp, Artificial sequence
  Synthesized
tctacgcgtaagcttgcacaaacgaacttc

SEQ ID NO: 91 - DNA, 55 bp, Artificial sequence
  Synthesized
tctatgcattaatgatagttaatgactaatagagtaaaacaagtcctcaagaggt

SEQ ID NO: 92 - DNA, 71 bp, Artificial sequence
  Synthesized
tgacataactaattacatgatctattagtcattaactatcattagatcagagtggggacgactaagaaagc

SEQ ID NO: 93 - DNA, 71 bp, Artificial sequence
  Synthesized
gctttcttagtcgtccccactctgatctaatgatagttaatgactaatagatcatgtaattagttatgtca

SEQ ID NO: 94 - DNA, 23 bp, Artificial sequence
  Synthesized
gcaaattaaagccttcgagcgtc

SEQ ID NO: 95 - DNA, 21 bp, Artificial sequence
  Synthesized
aagacccatgccgaacacgac

SEQ ID NO: 96 - DNA, 21 bp, Artificial sequence
  Synthesized
gctctgaggcaccttgggtaa

(19) United States
(12) Patent Application Publication
(10) Pub. No.: US 2023/0279337 A1
(43) Pub. Date: Sep. 7, 2023

(54) METHOD FOR CONSTRUCTING ENGINEERED YEAST FOR GLYCOPROTEIN PREPARATION AND STRAIN THEREOF

(71) Applicant: Academy of Military Medical Sciences, AMS, PLA, Beijing (CN)

(72) Inventors: Jun WU, Beijing (CN); Bo LIU, Beijing (CN); Peng SUN, Beijing (CN); Xin GONG, Beijing (CN); Tiantian WANG, Beijing (CN); Xuchen HOU, Beijing (CN); Bin ZHANG, Beijing (CN)

(21) Appl. No.: 17/996,710

(22) PCT Filed: Apr. 23, 2021
(86) PCT No.: PCT/CN2021/089158

§ 371 Date: Oct. 20, 2022

(30) Foreign Application Priority Data
Apr. 24, 2020 (CN) 202010331661.8

(51) Int. Cl. C12N 1/16; C12N 15/52

(52) U.S. Cl. CPC C12N 1/165; C12N 15/52

(57) ABSTRACT

The present invention discloses a method for constructing an engineered yeast for glycoprotein preparation and strains thereof. The present invention provides a method for constructing yeast engineered strain having the ability to modify a specific mammalian cell glycoform, comprising: inactivating endogenous α-1,6-mannose transferase, phosphomannose transferase, phosphomannose synthase, β-mannose transferase I-IV, and O-mannose transferase I of a receptore yeast; and expressing exogenous mannosidase I, N-acetylglucosamine transferase I, mannosidase II, N-acetylglucosamine transferase II, galactose isomerase and exogenous galactose transferase. The yeast engineered strain obtained in the present invention features a short construction period, fast growth, easy large-scale production, and high safety, so that they can not only be used to prepare common glycoprotein vaccines, but also very suitable for the efficient research and development and large-scale production of vaccines under emergency conditions such as sudden new infectious diseases. This has important implications in terms of medicinal uses.

### RELATED APPLICATIONS

The present application is a U.S. National Phase of International Application Number PCT/CN2021/089158, filed Apr. 23, 2021, and claims priority to Chinese Application Number 202010331661.8, filed Apr. 24, 2020.

### INCORPORATION BY REFERENCE

The sequence listing provided in the file entitled PUS1223197_SQL_rev1.txt, which is an ASCII text file that was created on Dec. 23, 2022, and which comprises 105,651 bytes, is hereby incorporated by reference in its entirety.

### FIELD OF THE INVENTION

The present invention relates to the field of bioengineering, in particular to a method for constructing an engineered yeast for glycoprotein preparation and a strain thereof.

### BACKGROUND OF THE INVENTION

As an important recombinant protein expression system, yeast has been widely used for the expression of various recombinant proteins. It has the advantages of fast growth like prokaryotic cell system, convenient gene manipulation and large-scale cultivation, and at the same time has the characteristics of eukaryotic post-translational processing and the ability to produce recombinant proteins with biological activity. Pichia pastoris (also known as Pichia pastoris) is a rapidly developing host strain for exogenous protein expression in recent years. In addition to the characteristics of general yeast, Pichia pastoris also has many advantages. For example, Pichia pastoris has a methanol-inducible promoter, which can strictly regulate the expression of exogenous proteins; the expression products of exogenous genes can present either intracellularly or be secreted extracellularly, and can efficiently obtain the products of exogenous genes. The expression vector can be inherited stably; it can be cultured for high-density and high-yield fermentation, which is convenient for industrial production, etc. In addition, it can also carry out many typical post-translational modifications of proteins in higher eukaryotes, such as glycosylation modification.

Glycosylation is critical for proper folding and stability of proteins. In the human body, glycosylation is one of the reasons that affect the pharmacokinetic properties of proteins (such as tissue distribution and blood clearance) (Guo Zhenchu, “Carbohydrate Chemistry”, Chemical Industry Press, 2005). There are two types of glycosyls in glycoproteins: N-glycosyl and O-glycosyl. The N- carbohydrate chain is connected to Asn in the Asn-X-Thr/Ser conservative sequence (where X is any amino acid residue except proline). The structure of O-carbohydrate chains is simpler than that of N- carbohydrate chains, and there are more connection sites than N-carbohydrate chains, which often appear on serine (Ser) and threonine (Thr). Glycosylation is critical for proper folding, stability, and biological activity of proteins. However, the glycosylation modification of yeast expressed proteins often produces excessive mannosylation, normal N-glycosyl modification, generally each glycosyl contains 10-20 monosaccharides, and the molecular weight is 1500-4000. In the case of excessive glycosylation modification, each glycosyl can contain tens to hundreds of mannoses, with a molecular weight of 5,000 to tens of thousands, which shows that the molecular weight of glycoprotein increases significantly, moreover, because the excessive glycosylation modification is often not uniform, the molecular weight of the glycoprotein is also not uniform, and there may be obvious “tailing” in SDS-PAGE analysis. N-glycosylation modification will occur at its conserved N-glycosylation modification site (N-X-S/T), but since O-glycosylation modification does not have a conserved glycosylation site, it is generally believed that it will occur on amino acids rich in serine or threonine, whether different proteins undergo O-glycosylation modification, and at which amino acid, the degree of O-glycosylation modification is different. Serine or threonine of protein may be a potential site of O-glycosylation, but not every serine or threonine will undergo O-glycosylation modification, and not every protein containing serine or threonine will undergo O-glycosylation modification, and the glycosylation modification of different proteins in different expression systems is also different. Glycoproteins with excessive mannosylation have a short half-life in the human body, are highly immunogenic, and are easily eliminated. Due to this defect, the use of Pichia pastoris in the production of most glycoprotein drugs is limited.

### SUMMARY OF THE INVENTION

The present invention aims to provide an engineered Pichia pastoris strain having the ability to modify a specific mammalian cell glycoform and a construction method thereof. The first technical problem to be solved is to construct yeast chassis cells, which require inactivation of glycosylation modification enzymes related to yeast itself, but due to the wide variety of glycosylation modification enzymes, the inactivation of many glycosyl modifications will kill the yeast, so this involves the uncertainty of the selection of modification enzymes. The second technical problem to be solved is to construct an engineered Pichia strain having the ability to modify a specific mammalian cell glycoform on yeast chassis cells, because eukaryotes have glycosylation modifications (in recent years, the phenomenon of glycosylation modification has also been found in prokaryotes), so there are many options for introducing glycosylation modification enzymes into yeast chassis cells, different species, different organelle localization methods, different temperature and pH regulation methods (because some organisms are resistant to cold, heat, acid, alkali, etc.), and different biological activities, these factors need to be considered, and a large number of combined experiments and analyzes need to be carried out through years of exploration and attempts. The present invention provides an engineered Pichia pastoris having the ability to modify a specific mammalian cell glycoform. The glycoform of the host protein expressed by the engineered yeast is a specific mammalian cell glycoform: GalₐGlcNAc_{b}Man_{c}GlcNAc₂, wherein a: 0-2; b: 0-2; c: 3-5 (Gal: galactose, GlcNAc: N-acetylglucosamine; Man: mannose); at the same time, the phenomenon of yeast O-glycosylation modification is further reduced.

In the first aspect, the present invention claims a method for constructing a Pichia pastoris engineered strain having the ability to modify a specific mammalian cell glycoform.

The method for constructing a Pichia pastoris engineered strain having the ability to modify a specific mammalian cell glycoform as claimed in the present invention may include the following steps:
(A1) inactivating the endogenous α-1,6-mannose transferase, phosphomannose transferase, phosphomannose synthase, β-mannose transferase I, β-mannose transferase II, β-mannose transferase III and β-mannose transferase IV of receptor Pichia pastoris, to obtain recombinant yeast 1;
(A2) expressing at least one of the following exogenous proteins in the recombinant yeast 1: exogenous mannosidase I, exogenous N-acetylglucosamine transferase I, exogenous mannosidase II, exogenous N- acetylglucosamine transferase II, exogenous galactose isomerase and exogenous galactose transferase to obtain recombinant yeast 2; the recombinant yeast 2 is a yeast engineered strain having the ability to modify a specific mammalian cell glycoform;
wherien the specific mammalian cell glycoform is GalₐGlcNAc_{b}Man_{c}GlcNAc₂, wherein a: 0-2; b: 0-2; c: 3-5(Gal: galactose, GlcNAc: N-acetylglucosamine; Man: mannose).

After inactivating α-1,6-mannose transferase, phosphomannose transferase, phosphomannose synthetase, and β-mannose transferase I-IV, the modification of N-glycosylation is significantly reduced, and the glycosyl internal environment tends to be relatively “clean”, a new question is: how to reduce O-glycosylation modification? There are many members of the O-glycosylation family, which enzyme inactivation may be applied to the present invention and achieve the desired effect? We all know that N-glycosylation modification will occur at its conservative N-glycosylation modification site (N-X-S/T), but since O-glycosylation modification has no conservative glycosylation site, it is generally believed that O-glycosylation modification occurs on amino acids enriched in serine or threonine, and whether O-glycosylation modification occurs in different proteins, and on which amino acid, the degree of O-glycosylation modification varies. Serine or threonine of protein may be a potential site of O-glycosylation, but not every serine or threonine will undergo O-glycosylation modification, and not every protein containing serine or threonine will undergo O-glycosylation modification, and the glycosylation modification of different proteins in different expression systems is also different. If O-glycosylation modification occurs, the glycosyl on the carbohydrate chain is mostly mannose. Although the carbohydrate chain is relatively short, due to the large number of carbohydrate chains, there may be a large amount of exposed mannose on the surface of the yeast expressed protein. This mannosylated glycoprotein has a short half-life in the human body, high immunogenicity, and is easy to be cleared. Due to this defect, the use of Pichia pastoris in the production of most protein drugs is limited.

According to the homology of O-glycosyl transferase family members, it is divided into three subfamilies: PMT1 subfamily, PMT2 subfamily and PMT4 subfamily. The number of members of PMT1 subfamily and PMT2 subfamily may be different in different species, and there are seven family members: PMT1\PMT2\PMT3\PMT4\PMT5\PMT6\PMT7. The PMT1 subfamily of Saccharomyces cerevisiae includes PMT1\PMT5\PMT7, and the PMT2 subfamily includes PMT2\PMT3\PMT638. Members of the Pmt1p subfamily (Pmt1p, Pmt5p) and Pmt2p subfamily (Pmt2p, Pmt3p) form heterodimers with each other, Pmt4p may form homodimers, and Pmt6p can neither form heterodimers with other members of the Pmtp family nor form homodimers with itself. In wild-type yeast, the complexes formed by members of the Pmt1p subfamily and Pmt2p subfamily are mainly Pmt1p-Pmt2p and Pmt5p-Pmt3p complexes, and there are also a small amount of Pmt1p-Pmt3p and Pmt2p-Pmt5p complexes. In the present invention, however, we found that on the basis of inactivation of α-1,6-mannose transferase, inactivation of phosphomannose transferase, inactivation of phosphomannose synthase and inactivation of β-mannose transferase I-IV, to further inactivate O- mannose transferase I, while expressing a certain type of exogenous mannosidase I, exogenous N-acetylglucosamine transferase I, exogenous mannosidase II, exogenous N-acetylglucosamine transferase II, exogenous galactose isomerase GalE and exogenous galactose transferase GalT, this combination can significantly reduce the O-glycosylation modification of proteins expressed by engineered yeast, and obtain specific mammalian cell glycoform.

Correspondingly, the method may also include the following steps (A3):

(A3) inactivating the endogenous O-mannose transferase I of the recombinant yeast 2 to obtain recombinant yeast 3; wherein the recombinant yeast 3 is also a yeast engineered strain having the ability to modify a specific mammalian cell glycoform.

Step (A3) further reduces the phenomenon of yeast O-glycosylation modification.

When the specific mammalian cell glycoform is Man₅GlcNAc₂, the exogenous protein expressed in the recombinant yeast 1 in step (A2) is exogenous mannosidase I.

When the specific mammalian cell glycoform is GlcNAcMan₅GlcNAc₂, the exogenous protein expressed in the recombinant yeast 1 in step (A2) is exogenous mannosidase I and exogenous N-acetylglucosamine transferase I.

When the specific mammalian cell glycoform is GalGlcNAcMan₅GlcNAc₂, the exogenous protein expressed in the recombinant yeast 1 in step (A2) is exogenous mannosidase I, exogenous N-acetylglucosamine transferase I, exogenous galactose isomerase and exogenous galactose transferase.

When the specific mammalian cell glycoform is GalGlcNAcMan₃GlcNAc₂, the exogenous protein expressed in the recombinant yeast 1 in step (A2) is exogenous mannosidase I, exogenous N-acetylglucosamine transferase I, exogenous galactose isomerase exogenous galactose transferase, and exogenous mannosidase II.

When the specific mammalian cell glycoform is Gal₂GlcNAc₂Man₃GlcNAc₂, the exogenous protein expressed in the recombinant yeast 1 in step (A2) is exogenous mannosidase I, exogenous N-acetylglucosamine transferase I, exogenous galactose isomerase and exogenous galactose transferase, exogenous mannosidase II, and exogenous N-acetylglucosamine transferase II.

In steps (A1) and (A3), inactivation of the above-mentioned glycosyl-modifying enzymes may be achieved by mutating one or more nucleotide sequences of the gene, or by deleting part or the entire gene sequence, or by inserting nucleotides to destroy the original reading frame and prematurely terminate protein synthesis, etc. to achieve the inactivation of the gene or the activity of the protein encoded by the gene. The above-mentioned mutations, deletions and insertion inactivations may be obtained by conventional methods such as mutagenesis and knockout. These methods have been reported in many literatures, such as J. Sambrook et al., “Molecular Cloning Experiment Guide” Second Edition, Science Press, 1995. Other methods known in the art may also be used to construct gene-inactivated yeast strains. Wherein, the better strain is obtained by knocking out the partial sequence of the mannose transferase gene. The partial sequence is at least larger than three bases, preferably larger than 100 bases, and more preferably includes more than 50% of the encoding sequence. The strain obtained by knocking out the partial sequence of the glycosyl-modifying enzyme gene is not easy to produce back mutation, and the stability of the strain is higher than that constructed by methods such as point mutation, which is more conducive to use in medical and industrial fields.

Knocking out the partial sequence of the glycosylation modifying enzyme gene may include: first constructing a plasmid to knock out the gene: the plasmid includes homologous arm sequences on both sides of the gene to be knocked out, and the two homologous arms should be selected on both sides of the target gene, the length of the homologous arms is at least greater than 200 bp, and the optimal size is between 500 bp and 2000 bp. Insertion inactivation may also be used to obtain an amino acid sequence that undergoes substitution and/or deletion and/or addition of one or several amino acid residues, resulting in a nucleotide sequence that has no functional activity, and is constructed into a plasmid. The plasmid also carries URA3 (orotidine-5′-phosphate decarboxylase) gene, or bleomycin, or hygromycin B, or Blasticidin or G418, etc. as selection markers. Nucleotide sequences encoding homologous arm fragments of the flanking regions, and nucleotide sequences of proteins whose functions are to be disrupted may be obtained from publicly available National Center for Biotechnology Information (NCBI). Using the PCR method, a certain length of flanking homologous regions required for inactivating genes is obtained by using the Pichia host genome as a template, including the upstream and downstream flanking homology of the gene encoding region of the target gene (whose sequence has been published in NCBI) respectively, and a suitable restriction site is added to the primer section. Obtaining polynucleotides according to the sequence may use methods well-known in the art, such as PCR (J. Sambrook et al., “Molecular Cloning Experiment Guide” second edition, Science Press, 1995.), RT-PCR method, artificial synthetic methods, genomic DNA and methods for constructing and screening cDNA libraries are available. If necessary, polynucleotides may be mutated, deleted, inserted, connected to other polynucleotides, etc. by methods known in the art. The upstream (5′) and downstream (3′) flanking region homologous arm fragments obtained respectively are fused, and various methods well known in the art may be used under the premise of keeping the size of the respective fragments unchanged, such as by overlapping PCR, and the standard molecular cloning process used is described by J. Sambrook et al. (J. Sambrook et al., “Molecular Cloning Experiment Guide” second edition, Science Press, 1995.). The nucleic acid containing the fusion fragment of the homologous arm sequence of the gene to be inactivated may be cloned into various vectors suitable for yeast by methods known in the art. Or use the restriction sites on the respective homologous arms to insert specific regions of the vector respectively. The standard molecular cloning procedure used is described by J. Sambrook et al. (J. Sambrook et al., “Molecular Cloning Experiment Guide” second edition, Science Press, 1995.). Construction of recombinant knockout plasmids. The original plasmid may be selected from expression vectors and shuttle vectors suitable for yeast, and may contain replication sites, screening markers, auxotrophic markers (URA3, HIS, ADE1, LEU2, ARG4), etc. The construction methods of these vectors have been described in many literatures Publicly available (e.g. J. Sambrook et al., Molecular Cloning Experiment Guide, Second Edition, Science Press, 1995), and may also be purchased from various companies (e.g. Invitrogen life technologies, Carlsbad, California 92008, USA), The preferred vectors used are pPICZ αA and pYES2 yeast expression vectors. The inactivated vectors are all shuttle plasmids, which are first replicated and amplified in E. coli, and then introduced into host yeast cells. The vectors should carry resistance marker genes or auxotrophic marker genes to facilitate the screening of later transformants.

The homologous regions on both sides of the gene to be inactivated (the upstream is called the 5′ arm, and the downstream is called the 3′ arm) are respectively constructed into the yeast vector to form a recombinant knockout vector. Further use the linearization site of the homologous arm to linearize the knockout vector, and transform it into Pichia pastoris or one of its modified forms by electrotransformation for cultivation. The nucleic acid required for transformation into host cells may be obtained by common methods, such as preparation of competent cells, electrotransformation, lithium acetate method, etc. (A. Adams et al., “Experimental Guide to Yeast Genetics Methods”, Science Press, 2000). Successfully transformed cells, that is, cells containing the homologous region of the gene to be knocked out, may be identified by well-known techniques, such as cell being collected and lysed, DNA extracted, and then genotype identified by PCR methods; and the previous selection of the correct phenotype may be achieved by screening for nutrient-deficient or resistance markers. Transformants with correct recombination once, after being cultured in yeast basic culture medium and coated on secondary recombination screening plates such as 5-fluoroorotic acid plates containing uracil, grow clones that are further genotyped for PCR identification. The transformants with the correct deletion of the expected gene encoding region are screened, respectively.

In a specific embodiment of the present invention, in step (A1), the inactivating endogenous α-1,6-mannose transferase, phosphomannose transferase, phosphomannose synthetase, β-mannose transferase I, β-mannose transferase II, β-mannose transferase III and β-mannose transferase IV of the receptor Pichia pastoris are all knocked out by homologous recombination.

In a specific embodiment of the present invention, in step (A2), expressing the exogenous protein in the recombinant yeast 1 is achieved by introducing a gene encoding the exogenous protein into the recombinant yeast 1.

Further, the gene encoding the exogenous protein is introduced into the recombinant yeast 1 in the form of a recombinant vector.

Further, both the gene encoding exogenous mannosidase I and the gene encoding exogenous mannosidase II are introduced into the recombinant yeast 1 twice.

In a specific embodiment of the present invention, in step (A3), inactivating the endogenous O-mannose transferase I of the recombinant yeast 2 is not achieved by the present invention in the traditional way of knocking out the gene, but by cleverly inactivating (destroying its corresponding nucleotide sequence by way of insertion inactivation) the gene encoding O-mannose transferase I in the genomic DNA of the recombinant yeast 2 by insertion.

In the present invention, specifically, the front end and the end of the target fragment of the O- mannose transferase I encoding gene in the genomic DNA of the recombinant yeast 2 are respectively equipped with different combinations of stop codons, and the terminator is installed after the stop codons (such as CYC1TT terminator) at the end. The target fragments with different combinations of stop codons installed at the front end and at the end are specifically fragment obtained by using the genomic DNA of Pichia pastoris JC308 as a template and using primers PMT1-IN-5 and PMT1-IN-3 for PCR-amplification.
PMT1-IN-5 : 5′ -tctatgcattaatgatagttaatgactaatagagtaaaacaagtcctcaagaggt-3′ (SEQ ID No.91);
PMT1-IN-3: 5′-tgacataactaattacatgatctattagtcattaactatcattagatcagagtggggacgactaagaaa gc-3′ (SEQ ID No.92).

The next technical problem is to construct an engineered Pichia strain with the ability of modifying glycoforms in mammalian cells in yeast chassis cells. There are many and complex glycosyl modification enzymes involved in glycosyl modification in mammalian cells. What kind of enzyme modification will obtain what kind of glycoform? And the proportional combination of the obtained glycoforms is unknown before the research. The present invention is realized through the following technical methods:

In step (A2), the exogenous mannosidase I is expressed and localized in the endoplasmic reticulum.

The exogenous mannosidase I is derived from trichoderma viride, and is fused with the endoplasmic reticulum retention signal HDEL at the C-terminus.

In step (A2), the exogenous N-acetylglucosamine transferase I is expressed and localized in the endoplasmic reticulum or medial Golgi apparatus.

The exogenous N-acetylglucosamine transferase I may be N-acetylglucosamine transferase I derived from mammals, such as human N- acetylglucosamine transferase I (GenBank NO NM 002406), Candida albicans N-acetylglucosamine transferase I (GenBank NO NW_139513.1), Dictyostelium N-acetylglucosamine transferase I (GenBank NO NC_007088.5), etc., and may be fused with endoplasmic reticulum or medial Golgi apparatus localization signal at the N- or C-terminus, such as ScGLS, ScMNS1, PpSEC12, ScMNN9, etc.; preferably derived from humans, and containing mnn9 localization signal.

In step (A2), the exogenous mannosidase II is expressed and localized in the endoplasmic reticulum or medial Golgi apparatus.

The exogenous mannosidase II may be mannosidase II derived from filamentous fungi, plants, insects, Java, mammals, etc., such as Drosophila mannosidase II (GenBank NOX77652), nematode mannosidase II (GenBank NO NM 0735941), human mannosidase II (GenBank NO U31520), etc.; expressed mannosidase II may be fused with endoplasmic reticulum or medial Golgi apparatus localization signal at the N-terminal or C-terminal, such as ScGLS, ScMNS1, PpSEC12, ScMNN9, etc., preferably derived from nematodes, and containing mnn 2 localization signal.

In step (A2), the exogenous N-acetylglucosamine transferase II is expressed and localized in the endoplasmic reticulum or medial Golgi apparatus.

The exogenous N-acetylglucosamine transferase II may be N-acetylglucosamine transferase II derived from mammals, such as human N-acetylglucosamine transferase II (GenBank NO Q10469), mouse N-acetylglucosamine transferase II(GenBank NO Q09326), etc.; expressed N-acetylglucosamine transferase II may be fused with endoplasmic reticulum or medial Golgi apparatus localization signal at the N-terminal or C-terminal, such as ScGLS, ScMNS1, PpSEC12, ScMNN9, etc., preferably derived from humans, and containing mnn 2 localization signal.

In step (A2), the exogenous mannosidase II is expressed and localized in the endoplasmic reticulum or medial Golgi apparatus.

The exogenous mannosidase II is derived from nematodes and contains mnn2 localization signal.

In step (A2), the exogenous galactose isomerase and the exogenous galactose transferase are expressed and localized in the endoplasmic reticulum or medial Golgi apparatus.

Both the exogenous galactose isomerase and the exogenous galactose transferase are derived from mammals, and are fused with an endoplasmic reticulum or medial Golgi apparatus localization signal at the N-terminal or C-terminal.

The exogenous galactose isomerase and the exogenous galactose transferase are fusion proteins, both of which are derived from humans, and share a kre2 localization signal.

The galactose transferase may be a galactose transferase derived from mammals, such as human β-1,4-galactose transferase(GenBank NO gi: 13929461), mouse β-1,4-galactose transferase GenBank NO NC_000081.6) etc. The expressed galactose transferase may be fused with an endoplasmic reticulum or medial Golgi apparatus localization signal at the N-terminal or C-terminal, such as ScKRE2, ScGLS, ScMNS1, PpSEC12, ScMNN9, etc. The galactose transferase of the present invention is derived from humans, and share a kre 2 localization signal;

The α-1,6-mannose transferase may be the following B1) or B2):
B1) a protein whose amino acid sequence is SEQ ID No.1;
B2) a protein having the same function as the amino acid sequence shown in SEQ ID No.1 through substitution and/or deletion and/or addition of one or several amino acid residues, or a protein having more than 99%, more than 95%, more than 90%, more than 85% or more than 80% homology with the amino acid sequence shown in SEQ ID No.1 and having the same function.

The phosphomannose transferase may be the following B3) or B4):
B3) a protein whose amino acid sequence is SEQ ID No.2;
B4) a protein having the same function as the amino acid sequence shown in SEQ ID No.2 through substitution and/or deletion and/or addition of one or several amino acid residues, or a protein having more than 99%, more than 95%, more than 90%, more than 85% or more than 80% homology with the amino acid sequence shown in SEQ ID No.2 and having the same function.

The phosphomannose synthase may be the following B5) or B6):
B5) a protein whose amino acid sequence is SEQ ID No.3;
B6) a protein having the same function as the amino acid sequence shown in SEQ ID No.3 through substitution and/or deletion and/or addition of one or several amino acid residues, or a protein having more than 99%, more than 95%, more than 90%, more than 85% or more than 80% homology with the amino acid sequence shown in SEQ ID No.3 and having the same function.

The P-mannose transferase I may be the following B7) or B8):
B7) a protein whose amino acid sequence is SEQ ID No.4;
B8) a protein having the same function as the amino acid sequence shown in SEQ ID No.4 through substitution and/or deletion and/or addition of one or several amino acid residues, or a protein having more than 99%, more than 95%, more than 90%, more than 85% or more than 80% homology with the amino acid sequence shown in SEQ ID No.4 and having the same function.

The P-mannose transferase II may be the following B9) or B10):
B9) a protein whose amino acid sequence is SEQ ID No.5;
B10) a protein having the same function as the amino acid sequence shown in SEQ ID No.5 through substitution and/or deletion and/or addition of one or several amino acid residues, or a protein having more than 99%, more than 95%, more than 90%, more than 85% or more than 80% homology with the amino acid sequence shown in SEQ ID No.5 and having the same function.

The P-mannose transferase III may be the following B11) or B12):
B11) a protein whose amino acid sequence is SEQ ID No.6;
B12) a protein having the same function as the amino acid sequence shown in SEQ ID No.6 through substitution and/or deletion and/or addition of one or several amino acid residues, or a protein having more than 99%, more than 95%, more than 90%, more than 85% or more than 80% homology with the amino acid sequence shown in SEQ ID No.6 and having the same function.

The P-mannose transferase IV may be the following B13) or B14):
B13) a protein whose amino acid sequence is SEQ ID No.7;
B14) a protein having the same function as the amino acid sequence shown in SEQ ID No.7 through substitution and/or deletion and/or addition of one or several amino acid residues, or a protein having more than 99%, more than 95%, more than 90%, more than 85% or more than 80% homology with the amino acid sequence shown in SEQ ID No.7 and having the same function.

The O-mannose transferase I may be the following B15) or B16):
B15) a protein whose amino acid sequence is SEQ ID No.8;
B16) a protein having the same function as the amino acid sequence shown in SEQ ID No.8 through substitution and/or deletion and/or addition of one or several amino acid residues, or a protein having more than 99%, more than 95%, more than 90%, more than 85% or more than 80% homology with the amino acid sequence shown in SEQ ID No.8 and having the same function.

The exogenous mannosidase I may be the following B17) or B18):
B17) a protein whose amino acid sequence is SEQ ID No.9;
B18) a protein having the same function as the amino acid sequence shown in SEQ ID No.9 through substitution and/or deletion and/or addition of one or several amino acid residues, or a protein having more than 99%, more than 95%, more than 90%, more than 85% or more than 80% homology with the amino acid sequence shown in SEQ ID No.9 and having the same function.

The exogenous N-acetylglucosamine transferase I may be the following B19) or B20):
B19) a protein whose amino acid sequence is SEQ ID No.10;
B20) a protein having the same function as the amino acid sequence shown in SEQ ID No. 10 through substitution and/or deletion and/or addition of one or several amino acid residues, or a protein having more than 99%, more than 95%, more than 90%, more than 85% or more than 80% homology with the amino acid sequence shown in SEQ ID No. 10 and having the same function.

The fusion protein consisting of the galactose isomerase and the galactose transferase may be the following B21) or B22):
B21) a protein whose amino acid sequence is SEQ ID No. 11;
B22) a protein having the same function as the amino acid sequence shown in SEQ ID No.11 through substitution and/or deletion and/or addition of one or several amino acid residues, or a protein having more than 99%, more than 95%, more than 90%, more than 85% or more than 80% homology with the amino acid sequence shown in SEQ ID No.11 and having the same function.

The mannosidase II may be the following B23) or B24):
B23) a protein whose amino acid sequence is SEQ ID No.12;
B24) a protein having the same function as the amino acid sequence shown in SEQ ID No.12 through substitution and/or deletion and/or addition of one or several amino acid residues, or a protein having more than 99%, more than 95%, more than 90%, more than 85% or more than 80% homology with the amino acid sequence shown in SEQ ID No.12 and having the same function.

The acetylglucosamine transferase II may be the following B25) or B26):
B25) a protein whose amino acid sequence is SEQ ID No.13;
B26) a protein having the same function as the amino acid sequence shown in SEQ ID No.13 through substitution and/or deletion and/or addition of one or several amino acid residues, or a protein having more than 99%, more than 95%, more than 90%, more than 85% or more than 80% homology with the amino acid sequence shown in SEQ ID No. 13 and having the same function.

The gene encoding the exogenous mannosidase I may be the following C1) or C2):
C1) a DNA molecule whose amino acid sequence is SEQ ID No.14;
C2) a DNA molecule having more than 99%, more than 95%, more than 90%, more than 85% or more than 80% homology with the nucleotide sequence shown in SEQ ID No.14 and encoding the exogenous mannosidase I, or a DNA molecule hybridizing to the DNA molecule defined by C1) under stringent conditions and encoding the exogenous mannosidase I.

The gene encoding the exogenous N-acetylglucosamine transferase I may be the following C3) or C4):
C3) a DNA molecule whose nucleotide sequence is SEQ ID No.15;
C4) a DNA molecule having more than 99%, more than 95%, more than 90%, more than 85% or more than 80% homology with the nucleotide sequence shown in SEQ ID No.15 and encoding the exogenous mannosidase I, or a DNA molecule hybridizing to the DNA molecule defined by C3) under stringent conditions and encoding the N-acetylglucosamine transferase I.

The gene encoding the fusion protein consisting of the galactose isomerase and the galactose transferase may be the following C5) or C6):
C5) a DNA molecule whose nucleotide sequence is SEQ ID No.16;
C6) a DNA molecule having more than 99%, more than 95%, more than 90%, more than 85% or more than 80% homology with the nucleotide sequence shown in SEQ ID No.16 and encoding the exogenous mannosidase I, or a DNA molecule hybridizing to the DNA molecule defined by C5) under stringent conditions and encoding the fusion protein.

The gene encoding the mannosidase II may be the following C7) or C8):
C7) a DNA molecule whose nucleotide sequence is SEQ ID No.17;
C8) a DNA molecule having more than 99%, more than 95%, more than 90%, more than 85% or more than 80% homology with the nucleotide sequence shown in SEQ ID No.17 and encoding the exogenous mannosidase I, or a DNA molecule hybridizing to the DNA molecule defined by C7) under stringent conditions and encoding the mannosidase II.

The gene encoding the N-acetylglucosamine transferase II may be the following C9) or C10):
C9) a DNA molecule whose nucleotide sequence is SEQ ID No.18;
C10) a DNA molecule having more than 99%, more than 95%, more than 90%, more than 85% or more than 80% homology with the nucleotide sequence shown in SEQ ID No.18 and encoding the exogenous mannosidase I, or a DNA molecule hybridizing to the DNA molecule defined by C9) under stringent conditions and encoding the N-acetylglucosamine transferase II.

Among the above-mentioned proteins, homology refers to the identity of amino acid sequences. The homology of amino acid sequences may be determined using homology search sites on the Internet, such as the BLAST webpage of the NCBI homepage. For example, it may be done in advanced BLAST2.1, by using blastp as the program, setting the Expect value to 10, setting all Filters to OFF, using BLOSUM62 as Matrix, and setting Gap existence cost, Per residue gap cost and Lambda ratio to 11, 1 and 0.85 (default value) and searching for the identity of a pair of amino acid sequences to calculate, and then the value of identity (%) may be obtained.

Among the above-mentioned genes, homology refers to the identity of amino acid sequences. The homology of amino acid sequences may be determined using homology search sites on the Internet, such as the BLAST webpage of the NCBI homepage. For example, it may be done in advanced BLAST2.1, by using blastp as the program, setting the Expect value to 10, setting all Filters to OFF, using BLOSUM62 as Matrix, and setting Gap existence cost, Per residue gap cost and Lambda ratio to 11, 1 and 0.85 (default value) and searching for the identity of a pair of amino acid sequences to calculate, and then the value of identity (%) may be obtained.

Among the above-mentioned proteins and genes, the above 95% homology may be at least 96%, 97%, 98% identity. The above 90% homology may be at least 91%, 92%, 93%, 94% identity. The above 85% homology may be at least 86%, 87%, 88%, 89% identity. The above 80% homology may be at least 81%, 82%, 83%, 84% identity.

Among the above-mentioned genes, the stringent conditions may be as follows: hybridization in a mixed solution of 7% sodium dodecyl sulfate(SDS), 0.5 M NaPO₄ and 1 mM EDTA at 50° C., and rinsing in 2 × SSC, 0.1% SDS at 50° C., and may also be: hybridization in a mixed solution of 7% SDS, 0.5 M NaPO₄and 1 mM EDTA at 50° C., and rinsing in 1 × SSC, 0.1% SDS at 50° C.; and may also be: hybridization in a mixed solution of 7% SDS, 0.5 M NaPO₄and 1 mM EDTA at 50° C., and rinsing in 0.5 × SSC, 0.1% SDS at 50° C.; and may also be: hybridization in a mixed solution of 7% SDS, 0.5 M NaPO₄and 1 mM EDTA at 50° C., and rinsing in 0.1 × SSC, 0.1% SDS at 50° C.; and may also be: hybridization in a mixed solution of 7% SDS, 0.5 M NaPO₄and 1 mM EDTA at 50° C., and rinsing in 0.1 × SSC, 0.1% SDS at 65° C.; and may also be: hybridization in a mixed solution of 6×SSC, 0.5% SDS at 65° C., and washing the membranes once with 2 × SSC, 0.1% SDS and once with 1×SSC, 0.1% SDS. And may also be: hybridization in a mixed solution of 6xSSC, 0.5% SDS at 65° C., and washing the membranes once with 2 × SSC, 0.1% SDS and once with 1×SSC, 0.1% SDS.

Information about all the glycosyl-modifying enzymes of the present invention may be obtained from the National Center for Biotechnology Information (NCBI) or published literature, and the functions and definitions of related enzymes may also be obtained from the literature. Even if it is the same strain or species, due to different sources, the amino acids of various enzymes may be slightly different, but their functions are basically the same. Therefore, the enzymes of the present invention may also include these variants.

In the second aspect, the present invention claims to protect the Pichia pastoris engineered strain constructed by the method described in the first aspect above.

Further, the Pichia pastoris engineered strain is a strain with a preservation number of CGMCCNo.19488 preserved in the China General Microbiological Culture Collection Center.

In the third aspect, the present invention claims the use of the Pichia pastoris engineered strain in the preparation of the target protein modified with the specific mammalian cell glycoform described in the second aspect above.

In the fourth aspect, the present invention claims a method for preparing the target protein modified with the specific mammalian cell glycoform.

The method for preparing a target protein modified with the specific mammalian cell glycoform as claimed in the present invention can include the following steps: introducing an encoding gene capable of encoding the target protein into the Pichia pastoris engineered strain described in the second aspect above to obtain a recombinant yeast engineered strain; cultivating the recombinant yeast engineered strain to prepare the target protein with the specific specific mammalian cell glycoform.

In a specific embodiment of the present invention, the target protein is specifically an anti-Her2 antibody.

### Preservation Instructions

Strain Latin name: Pichia pastoris
Reference biological material: GJK30
Proposed Taxonomic Name: Pichia pastoris
Preservation institution: China General Microbiological Culture Collection Center
Preservation institution abbreviation: CGMCC
Address: No. 3, Yard 1, West Beichen Road, Chaoyang District, Beijing,
Date of preservation: March 18,2020
Registration number of the preservation center: CGMCC No.19488

### BRIEF DESCRIPITION OF THE FIGURE

FIGS. 1A and 1B are the result of the och1 gene identification and the glycoform analysis in GJK01 strain. FIG. 1A is the identification result of och1 gene. M stands for Marker; 1: GJK01 strain (och1 has been knocked out); 2: X33 strain (och1 has not been knocked out). FIG. 1B is the result of DSA-FACE glycoform analysis of the antibody expressed by GJK01 strain (och1 knockout).

FIG. 2 is the identification result of pno1 gene. M stands for Marker; 1: GJK02 strain (pno1 has been knocked out); 2: X33 strain (pno1 has not been knocked out).

FIG. 3 is the identification result of mnn4b gene. M stands for Marker; 1: GJK03 strain (mnn4b has been knocked out); 2: X33 strain (mnn4b has not been knocked out).

FIG. 4 is the result of DSA-FACE glycoform analysis of GJK01, GJK02, and GJK03 strains (och1, pno1, and mnn4b have been knocked out).

FIG. 5 is the identification result of ARM2 gene. M stands for Marker; 1: GJK04 strain (ARM2 has been knocked out); 2: X33 strain (ARM2 has not been knocked out) .

FIG. 6 is the identification result of ARM1 gene. M stands for Marker; 1: GJK05 strain (ARM1 has been knocked out); 2: X33 strain (ARM1 has not been knocked out).

FIG. 7 is the identification result of ARM3 gene. M stands for Marker; 1: GJK07 strain (ARM3 has been knocked out); 2: X33 strain (ARM3 has not been knocked out).

FIG. 8 is the identification result of ARM4 gene. M stands for Marker; 1: GJK18 strain (ARM4 has been knocked out); 2: X33 strain (ARM4 has not been knocked out).

FIG. 9 is the result of DSA-FACE glycoform analysis of GJK18 strain.

FIGS. 10A and 10B are the result of TrmdsI gene identification and DSA-FACE glycoform analysis of W10 strain. FIG. 10A is the identification result of TrmdsI gene. M stands for Marker; 1: TrmdsI is introduced into W10 strain; TrmdsI is absent in X33 strain. FIG. 10B is the result of DSA-FACE glycoform analysis of W10 strain.

FIGS. 11A and 11B are the result of GnTI gene identification and DSA-FACE glycoform analysis of 1-8 strain. FIG. 11A is the identification result of GnTI gene. M stands for Marker; 1: GnTI is introduced into 1-8 strain; 2: GnTI is absent in X33 strain. FIG. 11B is the result of DSA-FACE glycoform analysis of 1-8 strain.

FIGS. 12A and 12B are the result of GalE-GalT gene identification and DSA-FACE glycoform analysis of 1-8-4 strain. FIG. 12A is the identification result of GalE-GalT gene. M stands for Marker; 1: GalE -GalT is introduced into 1-8-4 strain; 2: GalE-GalT is absent in X33 strain. FIG. 12B is the result of DSA-FACE glycoform analysis of 1-8-4 strain.

FIGS. 13A-13C are the result of mdsII gene and GnTII gene identification and DSA-FACE glycoform analysis of 52-60 and 150L2 strains. FIG. 13A is the identification result of MdsII gene. M stands for Marker; 1: MdsII is introduced into 52-60 strain; 2: MdsII is absent in X33 strain. FIG. 13B is the identification result of GnTII gene. M stands for Marker; 1: GnTII is introduced into 150L2 strain; 2: GnTII is absent in X33 strain. FIG. 13C is the result of DSA-FACE glycoform analysis of 52-60 strain.

FIG. 14 is the result of PMT1 insertional inactivation gene identification. M stands for Marker; 1: PMT1 is not inactivated in X33 strain; 2: GJK30(PMT1 is inactivated).

FIGS. 15A-15C the analysis result of the glycoform structure of GJK30 engineered strain. FIG. 15A is Gal2GlcNAc2Man3GlcNAc2 structure in the early stage less than 50%; FIG. 15B is Gal2GlcNAc2Man3GlcNAc2 structure obtained by the GJK30 engineered strain accounts for more than 60% of the glycoforms; FIG. 15C an enzymatic analysis of this glycoform by glycosidase(New England Biolabs, Beijing).

### DETAILED DESCRIPTION OF EMBODIMENTS

The following examples facilitate a better understanding of the present invention, but do not limit the present invention. The experimental methods in the following examples are conventional methods unless otherwise specified. The test materials used in the following examples, unless otherwise specified, are purchased from conventional biochemical reagent stores. Quantitative experiments in the following examples are all set up to repeat the experiments three times, and the results are averaged.

Unless defined otherwise, all technical and scientific terms used herein have the same meaning as commonly understood by one of ordinary skill in the art to which the present invention belongs. Exemplary methods and materials are described below, although methods and materials similar or equivalent to those described herein can also be used to carry out the present invention, as will be apparent to those skilled in the art. All publications and other references mentioned herein are incorporated by reference in their entirety. In case of inconsistency, the present specification, including definitions, shall prevail. The materials, methods, and examples are illustrative only and not limiting.

pPICZαA, pYES2 vector, X33, GS115 Pichia pastoris are products of Invitrogen.

Pichia pastoris GJK01 CGMCC No. 1853(recorded in the invention patent ZL200610164912.8, publication number is CN101195809, is Pichia pastoris with inactivated α-1,6-mannose transferase.

The Pyrobest enzyme, LA Taq enzyme, dNTPs, restriction endonuclease, T4 ligase, etc. used in the experiment are purchased from Dalian Bao Biological Engineering Co., Ltd., and the pfu enzyme, kit, and DH5α competent cells are the products purchased from Beijing TransGene Co., Ltd. Whole gene synthesis, nucleotide synthesis, primer synthesis, sequencing, etc. are provided by Shanghai Sangon Biotech Co., Ltd.

The sequence information of related modification enzymes involved in the following examples is shown in Table 1.

TABLE 1

Related modification enzymes involved in the present invention

| Serial number | Name | Genbank number or Reference |
|---|---|---|
| 1 | α-1,6-mannose transferase)(OCH1) | XM_002489551.1 |
| 2 | Phosphomannose transferase(PNO 1) | XM_002493793.1 |
| 3 | Phosphomannose synthase | XM_002490493.1 |
| 4 | β-mannose transferase I | XM_002490715.1 |
| 5 | β-mannose transferase II | XM_002493837.1 |
| 6 | β-mannose transferase III | XM_002493857.1 |
| 7 | β-mannose transferase IV | XM_002493838.1 |
| 8 | O-mannose transferase 1(PMT1) | XM_002491055.1 |
| 9 | Mannosidase I | Zhan Jie. Cloning, expression and activity identification of trichoderma viride α-1,2-mannosidase in Pichia pastoris [Master’s thesis]. |
| 10 | N-acetylglucosamine transferase I | NM 002406 |
| 11 | Mannosidase II | NM 0735941 |
| 12 | N-acetylglucosamine transferase II | NO Q10469 |
| 13 | Galactose isomerase | gi41522 |
| 14 | Galactose transferase | gi13929461 |

### Example 1. Construction of an Engineered Pichia Pastoris Strain Having the Ability to Modify a Specific Mammalian Cell Glycoform

### 1. Construction of a Yeast Strain with Phosphomannose Transferase Gene Inactivation

The basic strain used in the present invention was the GJK01 strain constructed by us in in the early stage, the preservation number was CGMCC No. 1853, and the authorized patent number of the strain was ZL200610164912.8. The strain was a Pichia pastoris strain with α-1,6-mannose transferase inactivation. The amino acid sequence of α-1,6-mannose transferase (OCH1) was shown in SEQ ID No.1.

The yeast strain GJK02 with inactivated phosphomannose transferase gene was obtained by partially knocking out the DNA molecule encoding the phosphomannose transferase shown in SEQ ID No.2 in Pichia pastoris GJK01, that is, the recombinant yeast obtained by knocking out the phosphomannose transferase gene in the genome of GJK01 yeast.

### 1. Construction of Phosphomannose Transferase Gene Inactivated Vector

The knockout plasmid pYES2-pno1 for knocking out the mannose transferase (PNO1) gene was obtained by inserting the gene fragment (SEQ ID No.20) corresponding to mannose transferase(PNO1) into the vector pYES2 between the KpnI and XbaI restriction sites. Wherein, the nucleotides 7-1006 of SEQ ID No. 20 from the 5 ′ end were the upstream homologous arm of the knockout mannose transferase (PNO1) gene fragment; the nucleotide 1015-2017 of SEQ ID No. 20 from the 5 ′ end was the downstream homologous arm of the knockout mannose transferase (PNO1) gene fragment.

Details were as follows:

The genomic DNA of Pichia pastoris X33 was extracted by the glass bead preparation method (A. Adams et al., “Experimental Guide to Yeast Genetics Methods”, Science Press, 2000), and the genomic DNA was used as a template to amplify the homologous arms on both sides of the mannose transferase gene, the homologous arms on both sides of PNO1 were about 1 kb respectively, and the encoding gene of about 1.4 kb was missing in the middle.

The primers used to amplify the homologous arm of the upstream flanking region of pno1(PNO1 5′ homologous arm) were PNO-5-5 and PNO-5-3. The primer sequences were:

5′-AGTGGTACCGCAGTTTAATCATAGCCCACTGC -3′(SEQ ID No.
21, the underlined part was Kpn I recognition site
);

5′-ATTCCAATACCAAGAAAGTAAAGTgcggccgcAAGTGGAACTG
GCGCACCGGT-3′(SEQ ID No.22, the underlined part wa
s Not I recognition site).

The primers used to amplify the homologous arm of the downstream flanking region of PNO1(PNO1 3′ homologous arm) were PNO-3-5 and PNO-3-3. The primer sequences were:

5′-ACCGGTGCGCCAGTTCCACTTgcggccgcACTTTACTTTCTTGGTAT
TGGAAT-3′(SEQ ID No.23, the underlined part was No
t I recognition site);

5′-TGTTCTAGATCCGAGATTTTGCGCTATGGAGC-3′(SEQ ID
No.24, theunderlined part was Xba I recognition si
te).

The PCR amplification conditions of the two homologous arms were as follows: after denaturation at 94° C. for 5 minutes, 30 cycles of denaturation at 94°Cfor 30 sec, renaturation at 55° C. for 30 sec, extension at 72° C. for 1 min 30 sec, and finally extension at 72° C. for 10 min; the target fragment size was about 1kb. The PCR products were purified and recovred by PCR product recovery purification kit(purchased from Dingguo Biotechnology Co., Ltd., Beijing). The overlap extension PCR was used to fuse the 5′ and 3′ homologous arms of PNO1(see J. Sambrook et al., “Molecular Cloning Guidelines”, Second Edition, Science Press, 1995). The PCR products of 5 ′ and 3 ′ homologous arms of PNO1 were used as templates, and PNO-5-5/PNO-3-3 was used as primers. The PCR amplification conditions were as follows : after denaturation at 94° C. for 5 min, 30 cycles of denaturation at 94° C. for 1 min, renaturation at 55° C. for 1 min, extension at 72° C. for 3 min 30 sec, and finally extension at 72° C. for 10 min; the target fragment size was about 2 kb. The PCR products were purified and recovred by PCR product recovery purification kit.

Kpn I/ Xba I double enzyme digestion (the restriction endonucleases used in this experiment were all from Bao Biological Engineering Co., Ltd., Dalian) PCR product, after digestion, the product was inserted into the vector pYES2 with the same double enzyme digestion treatment (Invitrogen Corp. USA), and ligated with T4 ligase at 16° C. overnight, transformed Escherichia coli DH5α, and screened positive clones on LB plates containing ampicillin (100 µg/ml). The plasmids of positive clones were identified by double digestion with Kpn I/Xba I. The recombinant vector with fragments around 4200bp and 2000bp named pYES2-pno1 was obtained, which was the knockout plasmid used to knock out the mannose transferase (PNO1) gene. The upstream and downstream homologous arms of the pno1 gene were verified to be correct by final sequencing.

### 2. Transformation of Pichia Pastoris with Knockout Plasmid

The knockout plasmid pYES2-pno1 was transformed into Pichia pastoris GJK01(recorded in the invention patent ZL200610164912.8, the publication number is CN101195809) by electrotransformation, the electrotransformation method was well known in the art (such as A. Adams et al., “Experimental Guide to Yeast Genetics Methods”, Science Press, 2000). Prior to electrotransformation, the knockout plasmid was first linearized with the BamH I restriction site upstream of the 5′ homologous arm, then electroporated into the prepared competent cells, and spread on MD culture medium containing arginine and histidine (YNB 1.34 g/100 mL, biotin 4×10⁻⁵ g/100 mL, glucose 2 g/100 mL, agar 1.5 g/100 mL, arginine 100 mg/ml, histidine 100 mg/ml). After the clones grow on the culture medium, several clones were randomly selected to extract the genome, and the PCR method was used to identify whether the knockout plasmid was correctly integrated into the target site on the chromosome. The two pairs of primers used in the PCR reaction were: the primer sequence PNO-5-5OUT outside the 5′ homologous arm of the PNO1 gene: 5′-GCAGTTTAATCATAGCCCACTGCTA-3′(SEQ ID No.25) and the primer sequence inner01 on the vector: 5′-AGCGTCGATTTTTGTGATGCTCGTCA-3′(SEQ ID No.26). The enzyme used in the PCR reaction was rTaq (Bao Biological Engineering Co., Ltd.), and the PCR amplification conditions were as follows: after denaturation at 94° C. for 5 min, 30 cycles of denaturation at 94° C. for 30 sec, renaturation at 55° C. for 30 sec, extension at 72° C. for 3 min, and finally extension at 72° C. for 10 min; The size of the PCR product band was analyzed by gel electrophoresis, and the band amplified by the primers at about 2.3 kb was a positive clone.

### 3. PCR Identification of Positive Engineered Strains

One of the positive clones was inoculated on YPD culture medium (10 g/L yeast extract, 20 g/L peptone, 20 g/L glucose), cultured on a shaker at 25° C. for 12 hours, and the bacterial solution was spread on adenine deficient 5-FOA culture medium (YNB 1.34 g/100 mL, biotin 4×10⁻⁵ g/100 mL, glucose 2 g/100 mL, agar 1.5 g/100 mL, arginine 100 mg/ml, histidine 100 mg/ml, uracil 100 mg/ml, 5-FOA 0.1%) (wherein, YNB was a yeast nitrogen source without amino acids, a product of Beijing Xinjingke Biotechnology Co., Ltd., 5-FOA was 5-fluorouracil, from Sigma-aldrich POBOX14508, St. Louis, MO 63178 USA), and cultured at 25° C.

After clones were grown on 5-FOA culture medium, the genomes of these clones were extracted and identified by PCR: the genome was used as a template to identify primers for the sequences PNO1-ORF01 and PNO1-ORF02 outside the homologous arm of the pno1 gene on the chromosome, and the primer sequence were:

PNO1-ORF01: 5′-GGGAAAGAAAACCTTCAATTT-3′(SEQ ID
No.27);

PNO1-ORF02: 5′-TACAAGCCAGTTTCGCAATAA-3′(SEQ ID
No.28).

At the same time, the PCR reaction system using the genome of the wild-type X33 strain (Invitrogen company) as a template was set as a control. The enzyme used in the PCR reaction was LA Taq (Bao Biological Engineering Co., Ltd.). The PCR amplification conditions were as follows: after denaturation at 94° C. for 5 min, 30 cycles of denaturation at 94° C. for 30 sec, renaturation at 55° C. for 30 sec, extension at 72° C. for 3 min, and finally extension at 72° C. for 10 min.

In order to identify whether the α-1,6-mannose transferase was knocked out, the present invention introduced a reporter protein after obtaining the GJK01 engineered strain. The present invention used the anti-Her2 antibody as the reporter protein, the construction method of the expression vector of the anti-Her2 antibody, and the transformation method of the vector had been disclosed in the patent application (publication number: CN101748145A). Using this method, the anti-Her2 antibody expression vector was transferred into the GJK01 host strain, and the GJK01-HL engineered strain expressing the anti-Her2 antibody was obtained. The DSA-FACE glycoform analysis method had been publicly reported “Liu Bo, et al. A method for analyzing oligosaccharide chains using DSA-FACE. Biotechnology Communications. 2008. 19(6). 885-888″

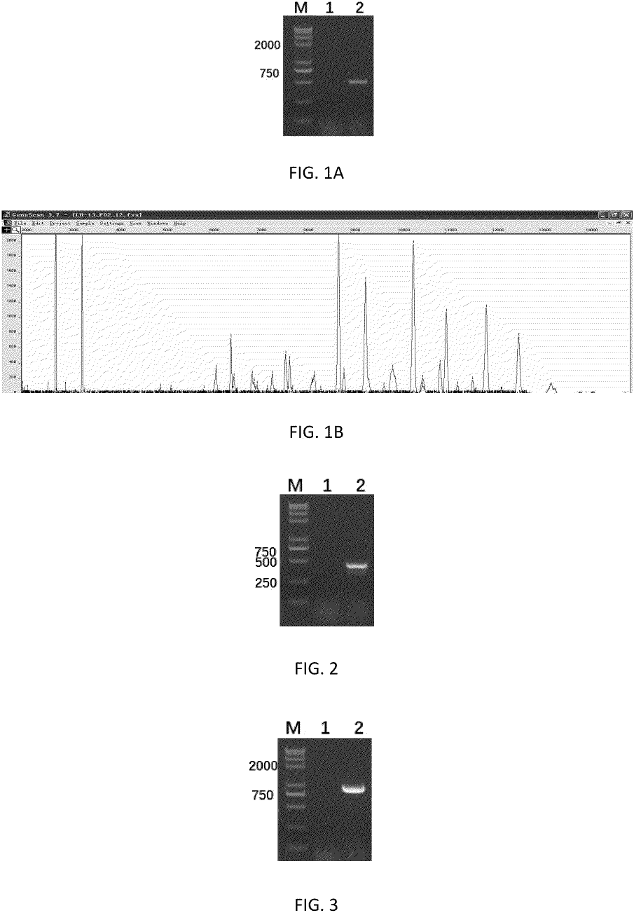

The product was subjected to agarose gel electrophoresis. A in FIG. 1 was the identification result of GJK01 host strain; B in FIG. 1 was the result of DSA-FACE glycoform analysis of GJK01-HL strain (och1 knockout). In FIG. 2, lane 1 was PON1-deficient, and lane 2 was wild-type; the size of the PCR product using the wild-type X33 strain genome as a template was about 490bp, and PON1-deficient engineered strain had no amplified bands, which also proved the loss of PNO1. The phosphomannose transferase knockout strain was constructed correctly, named GJK02, which was a recombinant Pichia pastoris with phosphomannose transferase knockout.

### II. Construction of a Yeast Strain with Phosphomannose Synthase Gene Inactivation

The yeast strain GJK03 with inactivated phosphomannose synthase gene was obtained by partially knocking out the DNA molecule encoding the phosphomannose synthase shown in SEQ ID No.3 in Pichia pastoris GJK02, that is, the recombinant yeast obtained by knocking out the phosphomannose synthase gene in the genome of GJK02 yeast. That is, theα-1,6-mannose transferase, phosphomannose transferase and phosphomannose synthase of the yeast were inactivated.

The method of constructing the vector was the same as step I.

### 1. Construction of Phosphomannose Synthase Gene Inactivated Vector

The knockout plasmid pYES2-MNN4B for knocking out the phosphomannose synthase gene was obtained by inserting the upstream and downstream homologous arms of the gene fragment corresponding to phosphomannose synthase into the vector pYES2 between the Stu I and Spe I restriction sites.

Using the same method as described in above-mentioned step I, the genomic DNA of Pichia pastoris X33 was extracted by the glass bead preparation method, and the genomic DNA was used as a template to amplify the knockout mannose synthase (MNN4B) gene fragment, the homologous arms on both sides of MNN4B were about 1 kb respectively, and the encoding gene of about 1 kb was missing in the middle.

The primers used to amplify the homologous arm of the upstream flanking region of MNN4B (ARM 2 5′ homologous arm) were MNN4B-5-5 and MNN4B-5-3. The primer sequences were:

5′-AGTAGGCCTTTCAACGAGTGACCAATGTAGA-3′(SEQ ID
No.29, theunderlined part was Stu I recognition si
te);

5′-TATCTCCATAGTTTCTAAGCAGGGCGGCCGCAATATGTGCGGT
GTAGGGAGAAA-3′(SEQ ID No.30, the underlined part w
as Not I recognition site).

The primers used to amplify the homologous arm of the downstream flanking region of MNN4B (MNN4B 3′ homologous arm) were MNN4B-3-5 and MNN4B-3-3. The primer sequences were:

5′-TTTCTCCCTACACCGCACATATTGCGGCCGCCCTGCTTAGAAA
CTATGGAGATA-3′(SEQ ID No.31, the underlined part w
as Not I recognition site);

5′-TGTACTAGTTGAAGACGTCCCCTTTGAACA-3′(SEQ ID No
.32, theunderlined part was Spe I recognition site
).

The PCR amplification conditions, recovery method, and enzyme digestion method of the two homologous arms were the same as in step 1, and the pYES2-MNN4B knockout vector was finally constructed and verified by final sequencing.

### 2. Transformation of Pichia Pastoris with Knockout Plasmid

The knockout plasmid was transformed into the Pichia pastoris engineered strain GJK02 constructed in above-mentioned step 1 by electrotransformation, and the electrotransformation method and identification method were the same as step I.

The two pairs of primers used in the PCR reaction were: the primer sequence MNN4B-5-5OUT outside the 5′ homologous arm of the mnn4b gene: 5′-TAGTCCAAGTACGAAACGACACTA-3′(SEQ ID No.33) and the primer sequence inner01 on the vector: 5′-AGCGTCGATTTTTGTGATGCTCGTCA-3′(SEQ ID No.26), the band amplified by the primer at about 2kb was a positive clone.

### 3. PCR Identification of Positive Engineered Strains

After growing clones by inoculating one of the positive clones on 5-FOA culture medium (the same formula as before), the genomes of these clones were extracted and identified by PCR: the genome was used as a template to identify primers for the sequences MNN4B-ORF01 and MNN4B-ORF02 outside the homologous arm of the mnn4B gene on the chromosome, and the primer sequence were:

MNN4B-ORF01: 5′-AAAACTATCCAATGAGGGTCTC-3′(SEQ
ID No.34);

MNN4B-ORF02: 5′-TCTTCAATGTCTTTAACGGTGT-3′(SEQ
ID No.35).

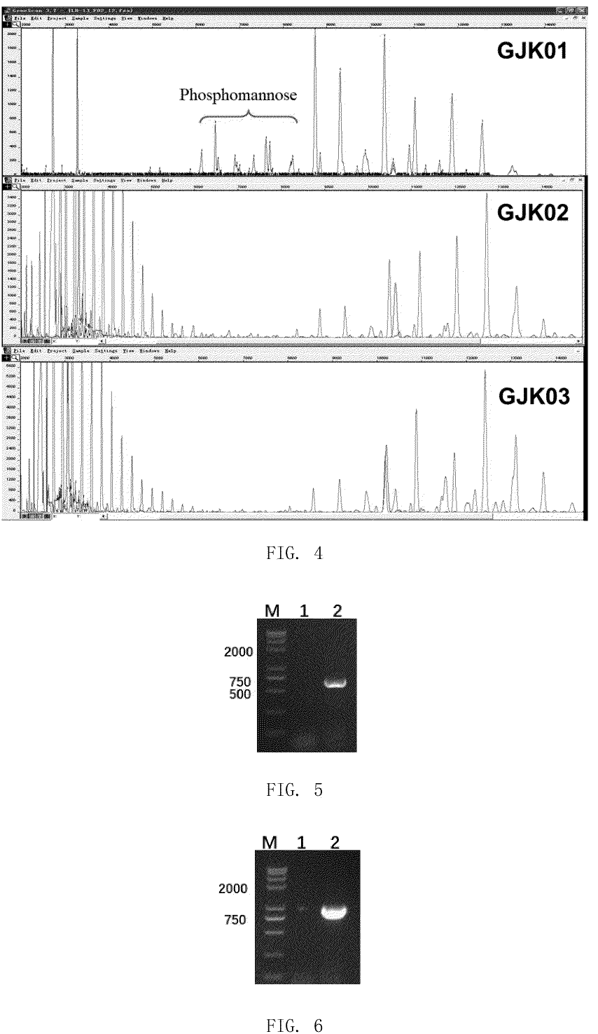

PCR amplification was performed using primers MNN4B-ORF01 and MNN4B-ORF02 with the positive clone genomic DNA as the template. The results were shown in FIG. 3. Lane 1 was MNN4B-deficient, and lane 2 was wild-type; the size of the PCR product using the wild-type X33 strain genome as a template was about 912bp, and MNN4-deficient engineered strain had no amplified bands, which also proved phosphomannose synthase knockout, named GJK03, which was the recombinant Pichia pastoris with phosphomannose transferase and phosphomannose synthase knockout.

The results of DSA-FACE glycoform analysis of GJK02 and GJK03 strains (which had knocked out och1, pno1, and mnn4b) (the method was the same as that described in Example 1) were shown in FIG. 4. It could be seen that the phosphomannose part of the glycoform was removed after pno1 and mnn4b knockout.

### III. Construction of a Yeast Strain with Β-Mannose Transferase Gene ARM2 Inactivation

The yeast strain GJK04 with inactivated phosphomannose transferase, phosphomannose synthase and β-mannose transferase ARM2 (i.e., β-mannose transferase II) genes were obtained by partially knocking out the DNA molecule encoding the β-mannose transferase ARM2 shown in SEQ ID No.5 in Pichia pastoris GJK03, that is, the recombinant yeast obtained by knocking out the β-mannose transferase ARM2 in the genome of GJK03 yeast; that is, the α-1,6-mannose transferase, phosphomannose transferase gene, phosphomannose synthase gene and β-mannose transferase ARM2 of the yeast genome were inactivated.

### 1. Construction of a Β-Mannose Transferase ARM2 Gene Inactivated Vector

The method of constructing the vector was the same as step I, details were as follows:

Using the same method as described in above-mentioned step I, the genomic DNA of Pichia pastoris X33 was extracted by the glass bead preparation method, and the genomic DNA was used as a template to amplify the homologous arms on both sides of the β-mannose transferase (ARM2), the homologous arms on both sides of ARM2 were about 0.6 kb respectively, and the encoding gene of about 0.6 kb was missing in the middle.

The primers used to amplify the homologous arm of the upstream flanking region of ARM2 (ARM 2 5′ homologous arm) were ARM2-5-5 and ARM2-5-3. The primer sequences were:

5′- ActTGGTACCACACGACTCAACTTCCTGCTGCTC-3′(SEQ
ID No.36, the underlined part was Kpn I recognitio
n site);

5′-actGCGGCCGCCACGAAACTTCTTACCTTTGACAA-3′(SEQ
ID No.37,the underlined part was Not I recognition
site).

The primers used to amplify the homologous arm of the downstream flanking region of ARM2 (ARM2 3′ homologous arm) were ARM2-3-5 and ARM2-3-3. The primer sequences were:

5′-TTGTCAAAGGTAAGAAGTTTCGTGGCGGCCGCTATCTTGACAT
TGTCATTCAGTGA-3′(SEQ ID No.38, the underlined part
was Not I recognition site);

5′-caaTCTAGAGCCTCCTTCTTTTCCGCCT-3′(SEQ ID No.3
9, the underlined part was Xba I recognition site)
.

### 2. Transformation of Pichia Pastoris with Knockout Plasmid

The knockout plasmid was transformed into the Pichia pastoris engineered strain GJK03 constructed in above-mentioned step 1 by electrotransformation, and the electrotransformation method and identification method were the same as step I.

The two pairs of primers used in the PCR reaction were: the primer sequence ARM2-5-5OUT outside the 5′ homologous arm of the ARM2 gene: 5′-TTTTCCTCAAGCCTTCAAAGACAG-3′(SEQ ID No.40) and the primer sequence inner01 on the vector: 5′-AGCGTCGATTTTTGTGATGCTCGTCA-3′(SEQ ID No.26), the band amplified by the primer at about 0.8 kb was a positive clone.

### 3. PCR Identification of Positive Engineered Strains

After growing clones by inoculating one of the positive clones on 5-FOA culture medium (the same formula as before), the genomes of these clones were extracted and identified by PCR: the genome was used as a template to identify primers for the sequences Arm-ORF01 and Arm-ORF02 outside the homologous arm of the ARM2 gene on the chromosome, and the primer sequence were:

Arm2-ORF-09: 5′-gggcagaagatcctagag-3′(SEQ ID N
o.41);

Arm2-ORF-10: 5′- tcgtctccattgctatctacgact -3′(
SEQ ID No.42).

PCR amplification was performed using primers Arm2-ORF-09 and Arm2-ORF-10 with the positive clone genomic DNA as the template. The results were shown in FIG. 5. Lane 1 was ARM2-deficient, and lane 2 was wild-type; the result was that the size of the PCR product using the wild-type X33 strain genome as a template was about 600bp, and ARM2-deficient engineered strain had no amplified bands, which also proved β-mannose transferase(ARM2) knockout, named GJK04, which was the recombinant Pichia pastoris with phosphomannose transferase, phosphomannose synthase and β-mannose transferase II(ARM) gene knockout.

### IV. Construction of a Yeast Strain With Β-Mannose Transferase ARM1, ARM3 and ARM4 Genes Inactivation

According to the above steps I to III, the design method and construction process of yeast strain construction with β-mannose transferase gene ARM2 inactivated, the β-mannose transferase ARM1, ARM3 and ARM4 genes were successively knocked out on the basis of GJK04 engineered strain (sequentially encoded β- mannose transferase I, III and IV respectively, and the amino acid sequences were SEQ ID No.4, SEQ ID No.6 and SEQ ID No.7 respectively), and the engineered strains GJK05, GJK07 and GJK18 were constructed respectively.

### 1. Construction of a Β-Mannose Transferase ARM1, ARM3, and ARM4 Genes Inactivated Vector

The method of constructing the vector was the same as step III, and the difference was:

The primers used to amplify the homologous arm of the upstream flanking region of ARM1 (ARM1 5′ homologous arm) were ARM1-5-5 and ARM1-5-3. The primer sequences were:

ARM1-5-5: 5′-TCAACGCGTTGGCTCTGGATCGTTCTAATA-3′
(SEQ ID No.43, the underlined part was MluI recogn
ition site);

ARM1-5-3: 5′ - ttctccgttctcctttctccgtGCGGCCGCc
agcagcaaggaagataccaa-3′ (SEQ ID No.44, the underli
ned part was NotI recognition site).

The primers used to amplify the homologous arm of the downstream flanking region of ARM1(ARM1 3′ homologous arm) were ARM1-3-5 and ARM1-3-3. The primer sequences were:

ARM1-3-5-. 5′- ttggtatcttccttgctgctgGCGGCCGCacg
gagaaaggagaacggagaa -3′(SEQ ID No.45, the underlin
ed part was NotI recognition site);

ARM1-3-3: 5′- TCAACGCGTTGGCTGGAGGTGACAGAGGAA -
3′(SEQ ID No.46, the underlined part was MluI reco
gnition site).

The primers used to amplify the homologous arm of the upstream flanking region of ARM3 (ARM 3 5′ homologous arm) were ARM3-5-5 and ARM3-5-3. The primer sequences were:

ARM3-5-5: 5′- TCAACGCGTTAGTAGTGCCGTGCCAAGTAGCG
-3′(SEQ ID No.47, the underlined part was MluI re
cognition site);

ARM3-5-3: 5′- tcctactttgcttatcatctgccGCGGCCGCg
gtcaggccctcttatggttgtg -3′(SEQ ID No.48, the under
lined part was NotI recognition site).

The primers used to amplify the homologous arm of the downstream flanking region of ARM3 (ARM3 3′ homologous arm) were ARM3-3-5 and ARM3-3-3. The primer sequences were:

ARM3-3-5: 5′-_cacaaccataagagggcctgaccGCGGCCGCgg
cagatgataagcaaagtagga -3′(SEQ ID No.49, the underl
ined part was Not I recognition site);

ARM3-3-3: 5′- TCAACGCGTCATAGGTAATGGCACAGGGATAG
-3′(SEQ ID No.50, the underlined part was MluIreco
gnition site).

The primers used to amplify the homologous arm of the upstream flanking region of ARM4 (ARM4 5′ homologous arm) were ARM4-5-5 and ARM4-5-3. The primer sequences were:

ARM4-5-5: 5′- TCAACGCGTGCAGCGTTTACGAATAGTGTCC
-3′(SEQ IDNo.51, the underlined part was MluI reco
gnition site);

ARM4-5-3: 5′- gcatagggctgaagcatactgtGCGGCCGCaa
tgatatgtacgttcccaaga -3′(SEQ ID No.52, the underli
ned part was NotI recognition site).

The primers used to amplify the homologous arm of the downstream flanking region of ARM4 (ARM4 3′ homologous arm) were ARM4-3-5 and ARM4-3-3. The primer sequences were:

ARM4-3-5: 5′-tcttgggaacgtacatatcattGCGGCCGCaca
gtatgcttcagccctatgc-3′(SEQ ID No.53, the underline
d part was NotI recognition site);

ARM4-3-3: 5′- TCAACGCGTGAGGTGGACAAGAGTTCAACAAA
G -3′(SEQID No.54, the underlined part was MluI re
cognition site).

### 2. Transformation of Pichia Pastoris with Knockout Plasmid

Same as Step III, the difference was that the two pairs of primers used in the PCR reaction were:

The primer sequence ARM1-5-5OUT outside the 5′ homologous arm of the ARM1 gene: 5′-GTTCTGGTATGCGTTCTA TTCTTC-3′(SEQ ID No.55) and the primer sequence inner01 on the vector: 5′-AGCGTCGATTTTTGTGATGCTCGTCA -3′(SEQ ID No.26), the band amplified by the primer at about 3.5 kb was a positive clone.

The primer sequence ARM3-5-5OUT outside the 5′ homologous arm of the ARM3 gene: 5′- TATTTGCCTTCTTCACCGT TAT-3′(SEQ ID No.56) and the primer sequence inner01 on the vector: 5′-AGCGTCGATTTTTGTGATGCTCGTCA-3′(SEQ ID No.26), the band amplified by the primer at about 3.7 kb was a positive clone.

The primer sequence ARMa4-5-5OUT outside the 5′ homologous arm of the ARM4 gene: 5′-TCCGTTGAGGGTGCTAAT GGTA-3′(SEQ ID No.57) and the primer sequence inner01 on the vector: 5′-AGCGTCGATTTTTGTGATGCTCGTCA-3′(SEQ ID No.26), the band amplified by the primer at about 3.7 kb was a positive clone.

### 3. PCR Identification of a Positive Engineered Strain

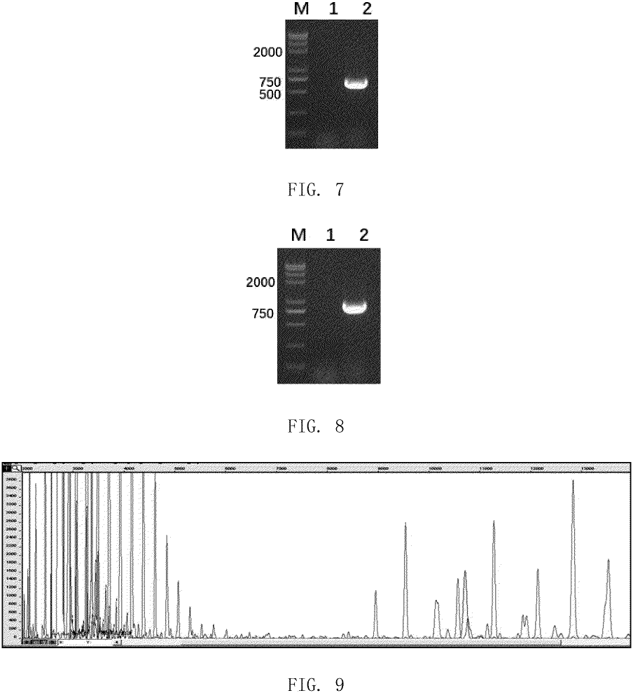

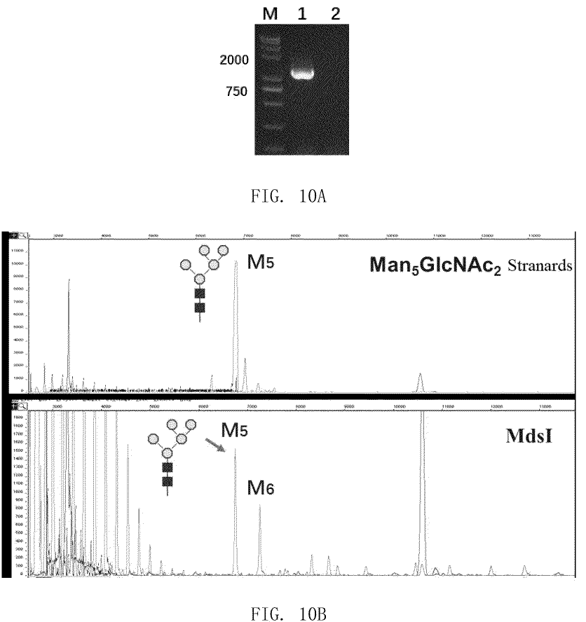

Same as Step III, the difference was that, using the following primers to identify the engineered strain, it could be found that the gene had been knocked out (FIG. 6, FIG. 7 and FIG. 8):

Arm1-ORF-09: 5′-TAGTCTGGTTTGCGGTAGTGT-3′(SEQ I
D No.58);

Arm1-ORF-10: 5′-AGATTGAGCATAGGAGTGGC-3′(SEQ ID
No.59).

Arm3-ORF-09: 5′-AAACGGAGTCCAGTTCTTCT-3′(SEQ ID
No.60);

Arm3-ORF-10: 5′-CAACTTTGCCTGTCATTTCC-3′(SEQ ID
No.61).

Arm4-ORF-09: 5′-CGCTTCAGTTCACGGACATA-3′(SEQ ID
No.62);

Arm4-ORF-10: 5′-GCAACCCAGACCTCCTTACC-3′(SEQ ID
No.63).

The results of DSA-FACE glycoform analysis of GJK18 strain were shown in FIG. 9. Because the modification of β-mannose was only added to individual ends of mannose, although the results of glycoform analysis had not changed substantially, β-mannose was a potential immunogenic sugar, so for the source of drugs used in humans, there were potential risks, and the present invention inactivated all β-mannose, thus fundamentally solving the problem of β-mannose, and the glycoform structure was not changed.

### V. Construction of a Glycoengineered Yeast Strain with Mammalian Man5GlcNAc2 and No Fucose Glycosylation Structure

First of all, in order to identify whether the exogenous mannosidase I (MDSI) played a role correctly, the present invention introduced a reporter protein in the GJK18 engineered strain in advance, and the present invention used the anti-Her2 antibody as the reporter protein, so the expression vector of anti- Her 2 antibody was constructed. The construction method of the vector and the transformation method of the vector had been disclosed in the patent application (publication number: CN101748145A). Using this method, the anti-Her2 antibody expression vector was transferred into the GJK18 host strain, and the W2 engineered strain expressing the anti-Her2 antibody was obtained.

Secondly, the glycoengineered yeast strain W10 with mammalian Man5GlcNAc2 and no fucose glycosylation structure was the engineered strain obtained by inserting MDSI (TrmdsI, nucleotide sequence as shown in SEQ ID No. 14, encoding the MDSI protein shown in SEQ ID No. 9) with C-terminal fusion HDEL sequence into the genome of the host strain W2.

### 1 Construction of an Exogenous Mannosidase I (MDSI) Expression Vector

The recombinant vector pPIC9-TrmdsI expressing exogenous mannosidase I was a recombinant vector obtained by inserting the DNA molecule shown in SEQ ID No.14 between the Xho I and EcoR I digestion sites of the pPIC9 vector.

Wherein, SEQ ID No.14 from the 5 ′ end of the 1-1524th nucleotide was the optimized mannosidase I encoding gene, and the 1525-1536th nucleotide from the 5 ′ end was the endoplasmic reticulum retention signal-HDEL encoding gene.

### Mannosidase I (MDSI) Gene

Exogenous mannosidase I could be mannosidase I derived from filamentous fungi, plants, insects, Java, mammals, etc., Mannosidase I (Zhan Jie. Cloning, expression and activity identification of trichoderma viride α-1,2-mannosidase in Pichia pastoris [ Master ’s thesis]) of trichoderma viride was selected for this example, and the endoplasmic reticulum retention signal-HDEL, was fused at the C-terminus of mannosidase I.

According to the mannosidase I sequence of trichoderma viride published by Zhan Jie “Cloning, expression and activity identification of trichoderma viride α-1,2-mannosidase in Pichia pastoris [ Master’s Degree ]”, the encoding gene was optimized according to the principle of yeast preference codon and the principle of high gene expression, and the gene fragment ( SEQ ID No.14) was obtained by fusing the HDEL sequence at the C-terminus.

### Design and Synthesize the Following Primers

TrmdsI-5: 5′-TCTCTCGAGAAAAGAGAGGCTGAAGCTTATCCAAAGC
CGGGC GCCAC-3′(SEQ ID No.64); the underlined part
was Xho I recognition site.

TrmdsI-3: 5′-AGGGAATTCTTACAACTCGTCGTGAGCAAGGTG
GCCGCCCCGT CGTGATG-3′ (SEQ ID No.65); the underlin
ed part was EcoR I recognition site.

The gene fragment obtained in the above-mentioneed (1) was used as a template, and TrmdsI-5 and TrmdsI-3 were used as primers to carry out PCR amplification to obtain a PCR amplification product, named TrmdsI, which contained SEQ ID No. 14.

The PCR products obtained from the above-mentioned (3) were doulbe digested by Xho I and EcoR I to obtain the gene fragment; pPIC9 vector was doulbe digested by Xho I and EcoR I to obtain large fragment of the vector. The gene fragment was ligated with the large fragment of the vector to obtain a recombinant plasmid, which was named pPIC9-TrmdsI. The pPIC9-TrmdsI was sequenced and the results were correct.

### 2. Construction of a Recombinant Yeast Expressing Exogenous Mannosidase I

About 10 µg pPIC9-TrmdsI plasmid was linearized with Sal I, and the linearized plasmid was precipitated with ⅒ volume of 3 M sodium acetate and 3 times the volume of anhydrous alcohol. The precipitate was washed twice with 70 % ethanol aqueous solution to remove salt, dried, and resuspended in about 30 µL water to obtain the linearized plasmid pPIC9-TrmdsI for transformation.

The method for preparing yeast electrotransformed competent cells in the following steps was based on Invitrogen’s relevant manual and “Molecular Cloning, A Laboratory Manual (Fourth Edition)”, 2012 Cold Spring Harbor Laboratory Press, Cold Spring Harbor, New YorK. The host strain selected was the W2 engineered strain constructed above.

Details were as follows:

Pichia pastoris W2 was isolated by streaking method on YPD plate (yeast extract 10 g/L, tryptone 20 g/L, glucose 20 g/L, agar 15 g/L), and cultured in a 28° C. incubator for 2 days. A single clone was inoculated into a 50 mL triangular flask containing 10 mL YPD liquid culture medium (yeast extract 10 g/L, tryptone 20 g/L, glucose 20 g/L) and cultured overnight at 28° C. until OD₆₀₀ was about 2. Then 0.1-0.5 mL bacterial solution was inoculated into a 3.5 L shake flask containing 500 mL YPD liquid culture medium and cultured overnight until OD₆₀₀ reached 1.3-1.5. The bacterial solution was transferred to sterile centrifugal bottle, 4° C., 1500 g centrifuge 10 minutes. The bacteria were resuspended with 500 mL of pre-cooled sterile water, and centrifuged at 4° C. for 10 minutes at 1500 g to harvest cells, and washed again with 250 mL of pre-cooled sterile water. The bactria were re-suspended with 20 mL pre-cooled sterile 1 M sorbitol, and centrifuged at 4° C., 1500 g for 10 minutes to harvest the cells. The bacteria were re-suspended with pre-cooled 1 M sorbitol to a final volume of 1.5 mL to obtain a bacterial suspension.

A total of 80 µL of bacterial suspension and 10 µL of pPIC9-TrmdsI linearized plasmid for transformation were mixed in a microcentrifugal tube to obtain a mixture, which was placed on ice for 5 min. The mixture was transferred to an ice-cold 0.2 cm electrotransformation cup and electroporated into cells (Bio-Rad Gene Pulser, 2000 V, 25 µF, 200 Ω). Then 1 mL of cold 1 M sorbitol was immediately added to the electrotransformation cup and the mixture (transformed cells) was carefully transferred to a 15 mL culture tube.

The culture tubes were incubated in a 28° C. incubator for 1 h without shaking. Then 1 mL YPD liquid culture medium was added and incubated in a shaker at 28° C., 250 rpm for 3 h. 200 µL of transformed cells were coated on a MD-containing plate (1.34 g / 100 ml YNB, 4×10⁻⁵ g /100 ml Biotin, 2 g/100 ml glucose). Incubated at 28° C. in a incubator for 2-5 days until the formation of a monoclonal, i.e. W2-Tr, named W10.

The genomic DNA of W10 was extracted by the glass bead preparation method, and the genomic DNA was used as a template, and TrMDSI-1.3 kb-01 and TrMDSI-1.3 kb-02 were used as primers to carry out PCR amplification to obtain a PCR amplification product of about 1.3 kb, proving MDSI had been inserted into the genome, which was a positive engineered strain(FIG. 10A).

TrMDSI-1.3kb-01: 5′-GAACACGATCCTTCAGTATGTA-3′
(SEQ ID No.66);

TrMDSI-1.3kb-02: 5′-TGATGATGAACGGATGCTAAAG-3′
(SEQ ID No.67).

The results of DSA-FACE glycoform analysis of W10 strain (the method was the same as that described in Example 1) were shown in FIG. 10B, it could be seen that the glycoform structure of the protein expressed by W10 strain after transferring into TrmdsI was Man5GlcNAc2 and Man6GlcNAc2, among which Man5GlcNAc2 was predominant.

### VI. Construction of Glycoengineered Yeast Strains with Mammalian GlcNAcMan5GlcNAc2 and No Fucose Glycosylation Structure

The glycoengineered yeast strain 1-8 with mammalian GlcNAcMan5GlcNAc2 and no fucose glycosylation structure was the engineered strain obtained by inserting a DNA fragment of N- acetylglucosamine transferase I (GnTI) (nucleotide sequence as shown in SEQ ID No. 15, encoding the protein shown in SEQ ID No. 10) containing an mnn9 localization signal into the genome of the host strain W10.

Wherein, the nucleotides 1-114 from the 5′ end of SEQ ID No. 15 were the mnn9 localization signal, and nucleotides 115-1335 from the 5′ end were the gene encoding N-acetylglucosamine transferase I.

### 1. Construction of a N-Acetylglucosamine Transferase I (GnTI) Expression Vector Containing mnn9 Localization Signal

### Retrieval of Human gnt1 Gene

The full-length fragment of human gnt1 gene was obtained from a human liver fetal cDNA library (purchased from Clontech Laboratories Inc. 1290 Terra Bella Ave. Mountain View, CA94043, USA) by PCR using human gnt1 gene upstream primer

(mnn9-GnTI-01: 5′-tcagtcagcgctctcgatggcgaccccg- 3′
(SEQ ID No.68)) and

downstream primer GnTI-02: 5′-

GCGAATTCTTAGTGCTAATTCCAGCTAGGATCATAG-3′( SEQ ID No
.69,

underlined was EcoRI restriction site). PCR reaction conditions : pre-denaturation at 94° C. 5 min, 30 cycles of denaturation at 94° C. for 30 sec, annealing at 52° C. for 30 sec, extension at 72° C. for 1 min 30 sec, and finally extension at 72° C. for 10 min. The PCR amplification products were separated by 0.8 % agarose gel electrophoresis and recovered by DNA recovery kit.

### GnTI DNA Fragment Containing the Localization Signal Mnn9

S. cere MNN9 Golgi localization signal: ScMNN9
-03:tatAAT attATGTCACTTTCTCTTGTATCGTACCGCCTAAGAAAGA
ACCCGTGGGTTAACATTTTTCTACCTGTTTTGGCCATATTTCTAATATAT
ATAATTTTTTTCCAGAGAGATCAATCTtcagtcagcgctctcgatggcga
ccccg(SEQ ID No.70)

The recovered purified 1.2-kb GnTI fragment was ligated to the S. cere MNN9 Golgi localization signal encoding sequence by PCR reaction using an upstream

primer ScMNN9-03(tatAATattATGTCACTTTCTCTTGTATCGTAC
CGCCTAAGAAAGAACCCGTGGGTTAACATTTTTCTACCTGTTTTGGCCAT
ATTTCTAATATATATAATTTTTTTCCAGAGAGATCAATCTtcagtcagcg
ctctcgatggcgaccccg(SEQ ID No.70),

underlined was the SspI digestion site)containing the S. cere MNN9 Golgi localization signal encoding sequence and a downstream primer GnTI-02 for the encoding region of the GnTI catalytic structural domain, and the mnn9-gnt1 gene fragment (SEQ ID No. 15) was amplified by using Pyrobest DNA polymerase.

PCR reaction conditions: denaturation at 94° C. for 2 min, annealing at 52° C. for 30 sec, extension at 72° C. for 5 min, followed by 30 cycles of denaturation at 94° C. for 30 sec, annealing at 52° C. for 30 sec, extension at 72° C. for 1 min 30 sec; finally extension at 72° C. for 10 min.

The PCR amplification products were separated by 0.8% agarose gel electrophoresis (8 V/cm, 15 min), and the 1.3-kb target bands were cut off with a clean blade under UV light and recovered with a DNA recovery kit. The method was the same as above.

### Construction of a PGE-URA3-GAP1-mnn9-GnTI Expression Vector

The mnn9-gnt1 gene fragment PCR products obtained from the above-mentioned (2) were doulbe digested by Ssp I and EcoR I to obtain the gene fragment; PGE-URA3-GAP1 vector (Yang Xiaopeng, Liu Bo, Song Miao, Gong Xin, Chang Shaohong, Xue Kuijing, Wu Jun. Construction of Pichia pastoris expression system for Man5GlcNAc2 mammalian mannose glycoprotein. Bioengineering Journal. 2011; 27: 108-17.) was doulbe digested by Ssp I and EcoR I to obtain large fragment of the vector. The gene fragment was ligated with the large fragment of the vector to obtain a recombinant plasmid, which was named PGE-URA3-GAP1-mnn9-GnTI. Sequenced and the results were correct.

PGE-URA3-GAP1-mnn9-GnTI was a recombinant vector obtained by inserting the DNA molecule shown in SEQ ID No.15 between the restriction site Ssp I and EcoR I of PGE-URA3-GAP1 vector.

### 2. Construction of a Recombinant Yeast Expressing Exogenous Mannosidase I

Approximately 10 µg of PGE-URA3-GAP1-mnn9-GnTI plasmid was linearized with Nhe I to obtain PGE-URA3-GAP1-mnn9-GnTI linearized plasmid for transformation to prepare yeast electrotransformed competent cells by the method as described aboved in step V.

The host strain selected was the W10 engineered strain constructed in step V above. The monoclonal formed on MD plates after transformation, named 1-8.

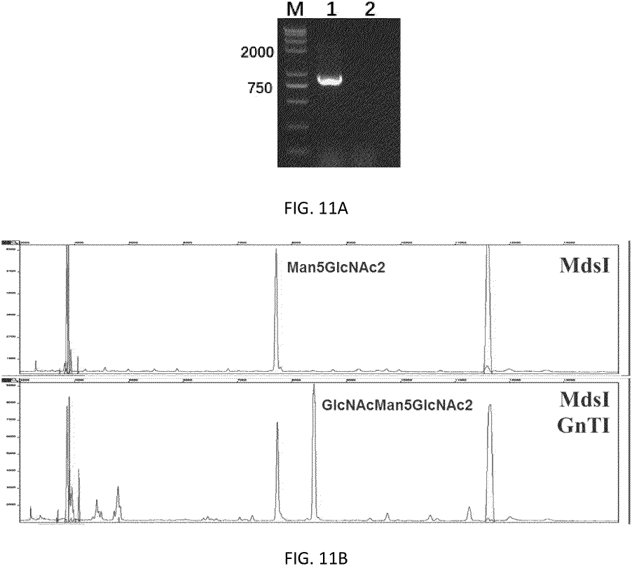

The genomic DNA of 1-8 was extracted by the glass bead preparation method, and the genomic DNA was used as a template, and HuGnTI-0.9k-01 and HuGnTI-0.9k-02 were used as primers to carry out PCR amplification to obtain a PCR amplification product of about 0.9 kb, proving GnTI had been inserted into the genome, which was a positive engineered strain (FIG. 11A).

HuGnTI-0.9k-01: 5′-TGGACAAGCTGCTGCATTATC-3′ (S
EQ ID No.71);

HuGnTI-0.9k-02: 5′-CGGAACTGGAAGGTGACAATA-3′ (S
EQ ID No.72).

The result of DSA-FACE glycoform analysis of 1-8 strain (the method was the same as that described in Example 1) was shown in FIG. 11B, it could be seen that the main glycoform structure of the protein expressed by the host strain after transferring into TrmdsI was GlcNAcMan5GlcNAc2.

### VII. Construction of a Glycoengineered Yeast Strain with Mammalian GalGlcNAcMan5GlcNAc2 and No Fucose Glycosylation Structure

The glycoengineered yeast strain 1-8-4 with mammalian GalGlcNAcMan5GlcNAc2 and no fucose glycosylation structure was the engineered strain 1-8-4 obtained by inserting kre2-GalE-GalT gene fragment (nucleotide sequence as shown in SEQ ID No. 16, encoding the protein shown in SEQ ID No. 11) into the genome of the host strain 1-8.

Wherein, the nucleotides 1-294 from the 5′ end of SEQ ID No. 16 were the kre2 localization signal, and nucleotides 295-1317 from the 5′ end were the gene encoding galactose isomerase GalE, the nucleotides 1325-2394 from the 5′ end were the gene encoding galactose transferase GalT.

### 1. Construction of a Galactose Transferase (GalE+T) Expression Vector Containing kre2 Localization Signal

### Retrieval of Human GalE and GalT Genes

The full-length fragment of human GalE and GalT genes were obtained from a human liver fetal cDNA library (purchased from Clontech Laboratories Inc. 1290 Terra Bella Ave. Mountain View, CA94043, USA) by PCR using human GalE gene upstream primer GalE5′, downstream primer GalE3′, the human GalT gene upstream primer GalT5′ and downstream primer GalT3′. PCR reaction conditions : pre-denaturation at 94° C. for 5 min, 30 cycles of denaturation at 94° C. for 30 sec, annealing at 52° C. for 30 sec, extension at 72° C. for 1 min 30 sec, and finally extension at 72° C. for 10 min. The PCR amplification products were separated by 0.8 % agarose gel electrophoresis and recovered by DNA recovery kit.

GalE5′: 5′ -ATGAGAGTTCTGGTTACCGGTGGTA-3′ (SEQ
ID No.73);

GalE3′ : 5′ -AGGGTACCATCGGGATATCCCTGTGGATGGC-3′(SE
Q IDNo.74, underlined part was Kpn I recognition s
equence);

GalT5′: 5′ -ATGGTACCGGTGGTGGACGTGACCTTTCTCGTCT
GCCA-3′(SEQ ID No.75, underlined part was Kpn I re
cognition sequence).

GalT3′ : 5′ - GCatttaaatttaGCTCGGTGTCCCGATGTCC
ACTGTGAT-3′(SEQID No.76, underlined part was SwaI
recognition sequence).

### GalE-GalT DNA Fragment Containing the Localization Signal Kre2

Kre2 5′ : 5′-ATAATattAAACGATGGCCCTCTTTCTCAGTAA
GAG-3′(SEQID No.77, underlined part was SspI I rec
ognition sequence);

Kre2 3′+GalE5′: 5′-CACCGGtAACCAGaACTctCatGATCG
GGGCAtctgccttttcagcggcagctttcagagccttggattc-3′( SE
Q ID No.78).

The kre2 localization signal fragment was retrieved from Saccharomyces cerevisiae S. cere genomic DNA by PCR. PCR conditions were the same as above.

The recovered purified GalE and GalT fragments were ligated to the S. cere kre2 Golgi localization signal encoding sequence by PCR reaction using an upstream primer Kre2 and a downstream primer GalT3′ for the encoding region of the GalE+GalT catalytic structural domain, and the kre2-GalE-GalT gene fragment was amplified by using Pyrobest DNA polymerase.

PCR reaction conditions: denaturation at 94° C. for 2 min, annealing at 52° C. for 30 sec, extension at 72° C. for 5 min, followed by 30 cycles of denaturation at 94° C. for 30 sec, annealing at 52° C. for 30 sec, extension at 72° C. for 4 min 30 sec; finally extension at 72° C. for 10 min.

The PCR amplification products were separated by 0.8% agarose gel electrophoresis (8 V/cm, 15 min), and the 2.4 kb target bands were cut off with a clean blade under UV light and recovered with a DNA recovery kit. The method was the same as above.

### Construction of PGE-URA3-GAP1-kre2-GaIE-GaIT Vector

The DNA molecule of the above kre2-GalE-GalT was first cleaved by SwaI enzyme, and then the gene fragment was phosphorylated by T4 PNK enzyme (Dalian Bao Biological Co., Ltd.); the PGE-URA3-GAP1 vector was double digested by Ssp I and SwaI to obtain the large fragment of the vector; the gene fragment was ligated with the large fragment of the vector to obtain the recombinant plasmid, which was named as PGE-URA3-GAP1-kre2-GalE-GalT. Sequenced and the results were correct.

PGE-URA3-GAP1-kre2-GalE-GalT was a recombinant vector obtained by inserting the DNA molecule of kre2-GalE-GalT shown in SEQ ID No. 16 between the restriction site Ssp I and SwaI of PGE-URA3-GAP1 vector.

### 2. Construction of Recombinant Yeast Expressing Exogenous UDP-Gal and Lactose Transferase

Approximately 10 µg of PGE-URA3-GAP1-kre2-GalE-GalT plasmid was linearized with Nhe I to obtain PGE-URA3-GAP1-kre2-GalE-GalT linearized plasmid for transformation to prepare yeast electrotransformed competent cells by the method as described aboved in step V.

The host strain selected was the 1-8 engineered strain constructed in step VI above. The monoclonal formed on MD plates after transformation, named 1-8-4.

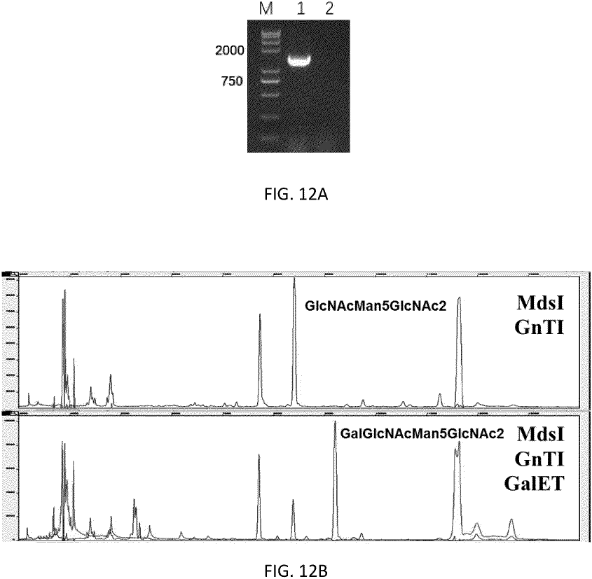

The genomic DNA of 1-8-4 was extracted by the glass bead preparation method, and the genomic DNA was used as a template, and GalE-T(1.5k)-01(5′-TGATAACCTCTGTAACAGTAAGCGC-3′, SEQ ID No.79) and GalE-T (1.5k)-02(5′-GGAGCTTAGCACGATTGAATATAGT-3′, SEQ ID No.80) were respectively used as primers for PCR amplification to obtain a PCR amplification product of 1.5 kb respectively, proving GalE-T had been inserted into the genome, which was a positive engineered strain (FIG. 12A).

The result of DSA-FACE glycoform analysis of 1-8-4 strain (the method was the same as that described in Example 1) was shown in FIG. 12B, it could be seen that the main glycoform structure of the protein expressed by the host strain after transferring into galactose isomerase and galactose transferase was GalGlcNAcMan5GlcNAc2.

### VIII. Construction of a Glycoengineered Yeast Strain with Mammalian GalGlcNAcMan3GlcNAc2 and No Fucose Glycosylation Structure

The glycoengineered yeast strain 52-60 with mammalian GalGlcNAcMan3GlcNAc2 and no fucose glycosylation structure was the engineered strain 52-60 obtained by inserting MDSII gene (nucleotide sequence as shown in SEQ ID No. 17, encoding the protein shown in SEQ ID No. 12) into the genome of the host strain 1-8-4.

Wherein, the nucleotides 1-108 from the 5′ end of SEQ ID No. 17 were the mnn2 localization signal of the gene encoding mannosidase II, and nucleotides 109-3303 from the 5′ end were the gene encoding mannosidase II.

### 1. Construction of a Mannosidase II(MDSII) Expression Vector Containing mnn2 Localization Signal

### Synthesis of a MDSII Gene Containing mnn2 Localization Signal by Whole Gene Synthesis

The MDSII gene containing mnn2 (SEQ ID No.17) was synthesized according to the sequence using whole gene synthesis, and was synthesized and cloned into the pUC57 cloning vector by Nanjing Jinris Company, to obtain pUC57-MDSII.

MDSII gene upstream primers (mnn2-MDSII-01: 5′-ATAATattAAACCatgctgcttaccaaaaggttttcaaagctgttc-3′, SEQ ID No.81)(underlined was SspI restriction site))and downstream primers (MDSII-02: 5′-GCTATTTAAATctattaCCTCAACTGGATTCGGAATGTGC TG ATTTCCATTG-3′, SEQ ID No.82)(underlined was SwaI restriction site)) were designed to obtain the PCR product of full-length fragment of human MDSII gene from pUC57-MDSII by PCR. PCR reaction conditions: pre-denaturation at 94° C. for 5 min, 30 cycles of denaturation at 94° C. for 30 sec, annealing at 52° C. for 30 sec, extension at 72° C. for 4 min 30 sec; final extension at 72° C. for 10 min. The PCR amplification products (sequence 17) were separated by 0.8% agarose gel electrophoresis and recovered with a DNA recovery kit.

### Construction of a PGE-URA3-arm3-GAP-mnn2-MDSII Expression Vector

The PCR product was first cleaved by SwaI enzyme, and then the gene fragment was phosphorylated by T4 PNK enzyme (Dalian Bao Biological Co., Ltd.); the PGE-URA3-GAP1 vector was double digested by Ssp I and SwaI to obtain the large fragment of the vector; the gene fragment was ligated with the large fragment of the vector to obtain the recombinant plasmid, which was named as PGE-URA3-arm3-GAP-mnn2-MDSII. Sequenced and the results were correct.

PGE-URA3-arm3-GAP-mnn2-MDSII was a recombinant vector obtained by inserting the DNA molecule shown in SEQ ID No. 17 between the restriction site Ssp I and SwaI of PGE-URA3-GAP1 vector.

### 2. Construction of a Recombinant Yeast Expressing Exogenous Mannosidase II

Approximately 10 µg of PGE-URA3-arm3--GAP-mnn2-MDSII plasmid was linearized with Msc I to obtain PGE-URA3-arm3-GAP-mnn2-MDSII linearized plasmid for transformation to prepare yeast electrotransformed competent cells by the method as described aboved in step V.

The host strain selected was the 1-8-4 engineered strain constructed in step VII above. The monoclonal formed on MD plates after transformation, named 52-60.

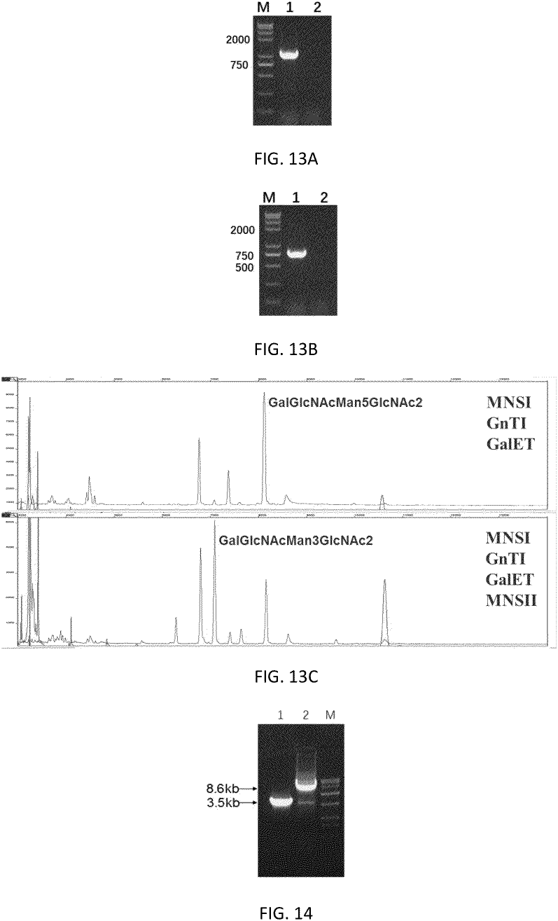

The genomic DNA of 52-60 was extracted by the glass bead preparation method, and the genomic DNA was used as a template, and CeMNSII-1.2k-01 and CeMNSII-1.2k-02 were respectively used as primers for PCR amplification to obtain a PCR amplification product of 1.2 kb respectively, proving MDSII had been inserted into the genome, which was a positive engineered strain (FIG. 13A).

CeMNSII-1.2k-01: 5′-CAGATGGATGAGCATAGAGTTA-3′(
SEQ ID No.83);

CeMNSII-1.2k-02: 5′-GACAAGAGGATAATGAAGAGAC-3′
(SEQ ID No.84).

The result of DSA-FACE glycoform analysis of 52-60 strain was shown in FIG. 13C, it could be seen that the main glycoform structure of the protein expressed by the host strain after transferring into exogenous mannosidase II was GalGlcNAcMan3GlcNAc2.

### IX. Construction of Glycoengineered Yeast Strains with Mammalian Gal2GlcNAc2Man3GlcNAc2 and No Fucose Glycosylation Structure

The glycoengineered yeast strain 150L2 with mammalian Gal2GlcNAc2Man3GlcNAc2 and no fucose glycosylation structure was the engineered strain 150L2 obtained by inserting GnT II DNA molecule (nucleotide sequence as shown in SEQ ID No. 18, encoding the protein shown in SEQ ID No. 13) into the genome of the host strain 52-60.

Wherein, the nucleotides 1-108 from the 5′ end of SEQ ID No. 18 were the mnn2 localization signal of the gene encoding N-acetylglucosamine transferase II, and nucleotides 109-1185 from the 5′ end were the gene encoding N-acetylglucosamine transferase II.

### 1. Construction of a N-Acetylglucosamine Transferase II(GnTII) Expression Vector for mnn2 Localization Signal

### Synthesis of GnTII Gene by Whole Gene Synthesis

The GnTII gene containing mnn2 (SEQ ID No.18) was synthesized according to the sequence using whole gene synthesis, and was synthesized and cloned into the pUC57 cloning vector by Nanjing Jinris Company, to obtain pUC57-GnTII.

GnTII gene upstream primers (mnn2-GnTII-01: 5′-ATAATattAAACCatgctgcttaccaaaa ggttttcaaagctgttc-3′, SEQ ID No.85)(underlined was SspI restriction site))and downstream primers (GnTII-02: 5′-GCTatttaaat TTAtcactgcagtcttctataacttttac-3′, SEQ ID No.86)(underlined was SwaI restriction site)) were designed to obtain the DNA molecule of N-acetylglucosamine transferase II(GnTII) containing mnn2 localization signal by PCR. PCR reaction conditions: pre-denaturation at 94° C. for 5 min, 30 cycles of denaturation at 94° C. for 30 sec, annealing at 52° C. for 30 sec, extension at 72° C. for 2 min 30 sec; final extension at 72° C. for 10 min. The PCR amplification products were separated by 0.8% agarose gel electrophoresis and recovered with a DNA recovery kit.

### Construction of a PGE-URA3-arm3-GAP-mnn2-GnTII Expression Vector

The method of enzyme digestion and construction was consistent with the construction method of PGE-URA3-arm3-GAP-mnn2-MDSII, and the recombinant plasmid was obtained, which was named as PGE-URA3-arm3-GAP-mnn2-GnTII. Sequenced and the results were correct.

PGE-URA3-arm3-GAP-mnn2-MDSII was a recombinant vector obtained by inserting the DNA molecule shown in SEQ ID No.18 between the restriction site Ssp I and SwaI of PGE-URA3-GAP1 vector.

### 2. Construction of a Recombinant Yeast Expressing Exogenous N-Acetylglucosamine Transferase II

Approximately 10 µg of PGE-URA3-arm3--GAP-mnn2-MDSII plasmid was linearized with Msc I to obtain PGE-URA3-arm3-GAP-mnn2-MDSII linearized plasmid for transformation to prepare yeast electrotransformed competent cells by the method as described aboved in step V.

The host strain selected was the 52-60 engineered strain constructed in step VIII above. The monoclonal formed on MD plates after transformation, named 150L2.

The genomic DNA of 150L2 was extracted by the glass bead preparation method, and the genomic DNA was used as a template, and RnGnTII-0.8k-01 and RnGnTII-0.8k-02 were respectively used as primers for PCR amplification to obtain a PCR amplification product of 0.8 kb, proving GnTII had been inserted into the genome, which was a positive engineered strain (FIG. 13B).

RnGnTII-0.8k-01: 5′-ATCAACAGTCTGATCTCTAGTG-3′
(SEQ ID No.87);

RnGnTII-0.8k-02: 5′-AGTTCATGGTCCCTAATATCTC-3′
(SEQ ID No.88).

### X. Knockout of Anti-her2 Antibody Gene in Engineered Strains

The yeast strain 3-5-11 with inactivated anti-her2 antibody gene was a recombinant yeast obtained by introducing the DNA molecule (anti-her2 antibody light-heavy chain gene knockout sequence) shown in SEQ ID No.19 into the Pichia pastoris 150L2, homologous recombination with the homologous sequence in the 150L2 genome, and knocking out the anti-her2 antibody light-heavy chain gene in the yeast genome.

The construction of anti-her2 antibody light-heavy chain gene inactivated vector, knockout plasmid for Pichia pastoris transformation, and PCR identification of positive engineered strain were performed in the same way as the previous steps, and the anti-her2 antibody gene inactivated yeast strain was named 3-5-11.

### XI. Inactivation of O-Mannose Transferase I Gene in Engineered Strains

Since the host strains were found to be unstable and prone to the loss of MDSI and MDSII genes, before the inactivation of O-mannose transferase I gene, the host strains were successively transferred into SEQ ID No. 17 (MDSII) and SEQ ID No. 14 (MDSI) in 3-5-11 according to the same technical method as in steps VIII and V of this Example, a double copy of these two genes in the engineered strain was ensured and the 670 host strain was obtained by the construction.

The yeast strain 7b with inactivated O-mannose transferase I gene was a yeast obtained by inactivating the DNA molecule encoding O- mannose transferase I shown in SEQ ID No. 8 by insertion in Pichia pastoris 670, named 7b, namely GJK30. GJK30 had been conserved in China General Microbiological Culture Collection Center on Mar. 18, 2020, with the conservation number of CGMCC No.19488.

### 1. Construction of an O-Mannose Transferase Gene Inactivation Vector

The terminator AOXTT sequence was obtained by PCR using plasmid pPIC9 (Invitrogen company) as a template. The PCR used to catch the terminator primers AOXTT-5 and AOXTT-3 (AOX1TT-5: 5′-tctacgcgtccttag acatgactgttcctcagt-3′(SEQ ID No.89); AOX1TT-3: 5′-tctacgcgtaagcttgcacaaacgaacttc-3′ (SEQ ID No.90)). The obtained PCR products were purified and recovered by PCR product recovery purification kit (Dingguo Biotechnology Co., Ltd., Beijing) to obtain the AOX1TT terminator fragment.

The vector pYES2 (Invitrogen company) used in the present invention had the URA3 screening marker for yeast and can be used for subsequent screening work. In order to prevent the promoter of URA3 gene on the vector from affecting other genes on the vector, the AOX1TT terminator was added at the end of URA3 gene by the present invention. The specific construction method was as follows: the AOX1TT terminator fragment obtained above was recovered and cleaved with MluI enzyme to obtain the enzyme fragment; the enzyme fragment was ligated with the vector pYES2 treated with the same Mlu1, and the ligated product was transformed into E. coli competent cells Trans5α (Beijing TransGene Co., Ltd., catalog number CD201) for amplification, and the clone with the correct sequence was named Trans5α-pYES2-URA3-AOX1TT, and the plasmid was extracted to obtain the recombinant vector with AOX1TT terminator added at the end of URA3 gene, which was recorded as pYES2-URA3-AOX1TT.

In order to enable the constructed vector to integrate into the PMT1 gene of Pichia pastoris in a targeted manner, PCR was used in the present invention to fish for a fragment of the ORF region of the PMT1 gene as a homologous recombinant fragment. In order to ensure that the integration of the inactivation vector into the PMT1 gene could cause inactivation of PMT1 gene, different combinations of stop codons were added at both ends of the primers, and the CYCTT terminator was added at the 3′ end of the fished PMT1 gene fragment.

The genomic DNA of Pichia pastoris JC308 was extracted by the glass bead preparation method (A. Adams et al., “Experimental Guide to Yeast Genetics Methods”, Science Press, 2000), and the genomic DNA was used as a template for PCR amplification using primers PMT1-IN-5 and PMT1-IN-3 to fish for the PMT1 gene fragment.

PMT1 -IN-5: 5′ -tctatgcattaatgatagttaatgactaat
agagtaaaacaagtcctcaagaggt-3′(SEQ ID No.91);

PMT1-IN-3: 5′ -tgacataactaattacatgatctattagtca
ttaactatcattagatcagagtggggacgactaagaaa gc-3′ (SEQ
IDNo.92).

The fished PMT1 gene fragment was joined at both ends with different combinations of stop codons and named as PMT1-IN.

The reaction conditions for PCR fishing for PMT1 gene fragment were pre-denaturation at 94° C. for 5 min, denaturation at 94° C. for 30 sec, annealing at 55° C. for 30 sec, and extension at 72° C. for 1 min and 40 s. A total of 25 cycles were performed, and final extension at 72° C. for 10 min. The recovered PCR product was the fished PMT1 gene fragment.

The plasmid pYES2 containing the CYCTT terminator was used as a template, and the CYC1TT terminator fragment was fished by PCR amplification using primers CYC1TT-5 and CYC1TT-3 (CYC1TT-5: 5′-gctttcttagtcgtccccactctgatctaatgatagttaatgactaatagatcatgtaattagttatgtca-3′ (SEQ ID No.93); CYC1TT-3: 5′-gcaaattaaagccttcgagcgtc-3′ (SEQ ID No.94)). PCR reaction conditions were pre-denaturation at 94° C. for 5 min, denaturation at 94° C. for 30 sec, annealing at 55° C. for 30 sec, and extension at 72° C. for 1 min. A total of 25 cycles were performed, and final extension at 72° C. for 10 min. The PCR product was recovered, which was namely the CYC1TT terminator fragment.

The recovered PCR product CYC1TT terminator fragment and PMT1-IN fragment (fished PMT1 gene fragment) were then used as templates, and PCR amplification was performed using primers PMT1-IN-5 and CYC1TT-3 to ligate PMT1-IN and CYC1TT fragments to construct a PMT1-IN-CYC1TT fusion fragment. The PCR reaction conditions were: pre-denaturation at 94° C. for 5 min, denaturation at 94° C. for 30 sec, annealing at 55° C. for 30 sec, and extension at 72° C. for 2.4 min. A total of 25 cycles were performed, and the final extension at 72° C. for 10 min. the PCR product was recovered as the linker fragment of PMT1-IN and CYC1TT terminator -PMT1-IN-CYC1TT fusion fragment. The recovered product was digested with Nsi1 and phosphorylated, and then ligated with the vector backbone obtained by Nsi1 and Stu1 digestion of pYES2-URA3-AOX1TT, and the recombinant vector with the correct sequence was obtained as PMT1 insertion inactivation vector PMT1-IN-pYES2.

Different combinations of stop codons were installed at the front and end of the fished PMT1 gene fragment, and a CYC1TT terminator was installed after the stop codon at the end to ensure that the PMT1 gene would not be expressed if the genome was integrated correctly. The pYES2 vector contained the URA3 gene of Pichia pastoris. In order to prevent the URA3 gene promoter from starting the PMT1 gene, an AOX1TT terminator was inserted behind the URA3 gene. According to the designed primers, a CYC1TT terminator (272bp) fragment and a PMT1 (907bp) fragment were obtained, which were consistent with the theoretical size. The size of the PMT1-IN fragment and CYC1TT fusion fragment was 1135bp, and the above PCR identification and sequencing proved that the vector PMT1-IN-pYES2 was successfully constructed.

### 2. Construction of a PMT1 Gene Inactivated Strain

Preparing Yeast 670 competent cells, and the preparation method was:

A single colony of 670 was picked and inoculated in 2 mL of YPD+U culture medium (which was a culture medium with uracil concentration of 100 µg/mL obtained by adding uracil to YPD culture medium) and incubated at 25° C. in a shaker at 170r/min for 48 h; then 500 µL of the culture was taken and inoculated in 100 mL of YPD+U culture medium and incubated at 170r/min for 24 h at 25° C., OD₆₀₀ reached 1.0; then centrifuged at 6000 r/min for 6 min at 4° C., and the strain was resuspended with 15 mL of cold sterile water; centrifuged again under the same conditions, and the strain was resuspended with 15 mL of cold sterile water; centrifuged at 6000 r/min for 6 min at 4° C., and the strain was resuspended with 15 mL of cold 1 mol/L sorbitol; centrifuged again under the same conditions. The supernatant was poured off and the strain was resuspended with 1 mL of cold 1 mol/L sorbitol in a volume of about 1.5 mL, i.e. yeast 670 competent cells, placed on ice for later use.

Electrotransformation transformation of PMT1 insertion inactivation vector PMT1-IN-pYES2: PMT1 insertion inactivation vector PMT1-IN-pYES2 was recovered after linearization using EcoRV digestion, and the final product was dissolved in 20 µL of ddH2O, which was the linearized plasmid; 85 µL of 670 competent cells were mixed with the linearized plasmid in an electrotransformation cup, placed on ice for 5 min, and electrotransformation (2 kV) was performed according to the conditions described in the handbook of electrotransformation of Pichia pastoris, immediately after electroshock, 700 µL of 1 M sorbitol was added, transferred to a 1.5 mL centrifuge tube, placed for 1 h at 25° C., coated on MD+RH plates (the plates were solid culture media with histidine and arginine concentrations of 100 µg/mL and 100 µg/mL, respectively, obtained by adding histidine and arginine to MD culture medium), incubated at 25° C., and the genomic DNA was extracted from the clones that grew on the plates, and PCR identification was done using the PMT1 genomic peripheral primers PMT1-ORF-OUT-5 and PMT1-ORF-OUT-3, and the clone with correct genomic identification was named 7b, i.e. GJK30.

PMT1-ORF-OUT-5: 5′-aagacccatgccgaacacgac-3′ (S
EQ ID No.95);

PMT1-ORF-OUT-3: 5′-gctctgaggcaccttgggtaa-3′ (S
EQ ID No.96).

The insertion of the insertion inactivation vector was used to integrate into the Pichia pastoris chromosome. Since the vector contained a homologous fragment of the PMT1 gene, the integration of the vector is theoretically a targeted integration, i.e., the insertion is on the PMT1 gene and could be identified and screened by specific primers designed. The clones grown on MD+RH plates were identified by stress screening using the URA3 screening marker of Pichia pastoris. PCR identification was done by the PMT1 gene peripheral primers PMT1-ORF-OUT-5 and PMT1-ORF-OUT-3. If the PMT1-IN-pYES2 vector was correctly integrated into the PMT1 gene, a fragment of 8.6 kb in size could be obtained using the above primers; the control (i.e. yeast X33) was a fragment of 3 kb in size (FIG. 14); it could be seen from that the PMT1-IN-pYES2 vector was correctly integrated into the PMT1 gene named 7b, i.e. GJK30. Since different stop codons and terminators were designed on the insertion vector, the PMT1 gene was not expressed when the gene was integrated correctly.

### XII. Analysis of the Glycoform Structure of GJK30 Engineered Strain

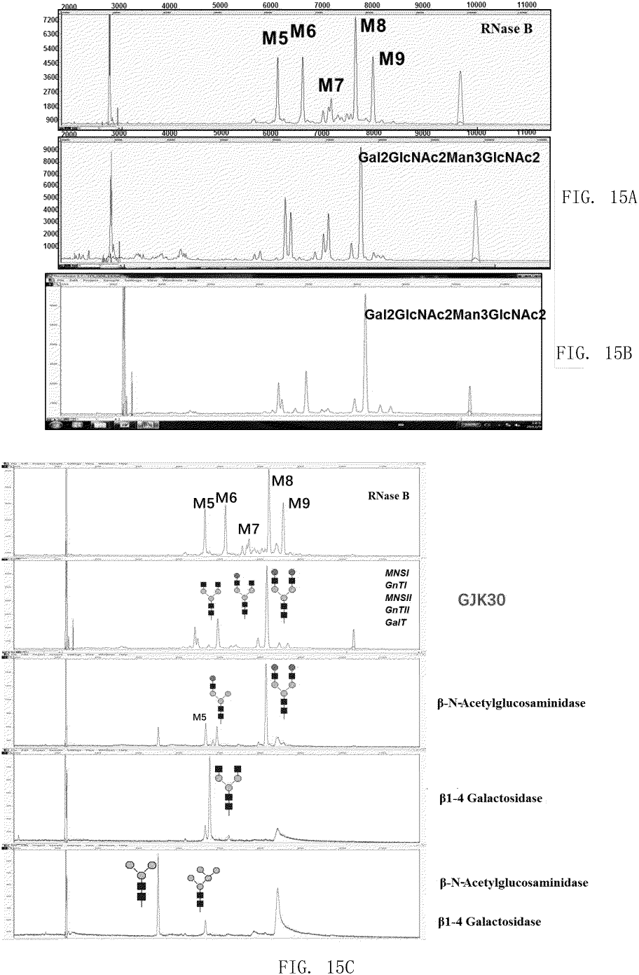

In order to observe whether the glycoform structure of GJK30 finally obtained was correct, a reporter protein was introduced after obtaining GJK30 engineered strain by the present invention, and the same method as in Example I, anti-Her2 antibody used as the reporter protein, the method of constructing the expression vector of anti-Her2 antibody and the transformation method of the vector had been disclosed in the patent application (see Example I). Using this method, the anti-Her2 antibody expression vector was transferred into the GJK30 host strain, and the GJK30-HL engineered strain expressing anti-Her2 antibody was obtained. Although both the glycoform type and the glycoform type obtained in the early stage (the control recombinant engineered strain obtained by transferring the Her2 antibody expression vector into the GJK08 strain constructed in Example 1 of Chinese patent application 201410668305.X, i.e., compared with the GJK30-HL engineered strain of the present invention, there were three differences: the β- mannose transferase knocked out in the present invention was I-IV, and the control recombinant engineered strain only knocked out β-mannose transferase II, the present invention also inactivated O-mannose transferase I, which was not present in the control recombinant engineered strain; the present invention introduced exogenous MDSI and MDSII twice, while the control recombinant engineered strain introduced them once) contained the Gal2GlcNAc2Man3GlcNAc2 structure, the proportion of the two glycoforms was obviously different, The Gal2GlcNAc2Man3GlcNAc2 structure in the early stage was less than 50% (FIG. 15A), while the Gal2GlcNAc2Man3GlcNAc2 structure obtained from GJK30 engineered strain accounted for more than 60% of the glycoforms, and the overall glycoforms were simpler and more uniform (FIG. 15B). According to numerous literature reports, this Gal2GlcNAc2Man3GlcNAc2 glycoform structure affected the biological activities of the protein, such as the ADCC and CDC activities of antibodies, and thus its proportion directly affected many properties of the protein. The glycoform was analyzed by enzymatic cleavage with a commercially purchased glycosidase (New England Biolabs, Beijing), as shown in FIG. 15C. Since Gal2GlcNAc2Man3GlcNAc2(G2) did not have N-acetylglucosamine at the end, in the presence of β-N-acetylglucosaminidase, Gal2GlcNAc2Man3GlcNAc2 structure would not change, but could shear off two galactoses by the action of exonuclease β1,4-galactosidase, and formed the structure of GlcNAc2Man3GlcNAc2 (G0); and at the same time under the action of these two exonucleases, that is, successively sheared off galactose Gal and N-acetylglucosamine GlcNAc, and thus the glycosyl structure changed to the Man3GlcNAc2 structure, proving that the expressed glycoforms were correct.

### INDUSTRIAL APPLICATION

The Pichia pastoris engineered strain obtained in the present invention has its own N-glycosyl and O-glycosyl reduced, and it has the ability to modify animal cell glycoforms, and the glycoproteins prepared by this engineered yeast strain avoid problems such as fungal-type glycosylation modification that may cause allergy, etc. The engineered Pichia pastoris strain features a short construction period, fast growth, easy large-scale production, and high safety, so that they can not only be used to prepare common glycoprotein vaccines, but also very suitable for the efficient research and development and large-scale production of vaccines under emergency conditions such as sudden new infectious diseases. This has important implications in terms of medicinal uses.

1-49. (canceled)

50. A method for constructing a Pichia pastoris engineered strain having the ability to modify a specific mammalian cell glycoform, comprising the following steps:
(A1) inactivating the endogenous α-1,6-mannose transferase, phosphomannose transferase, phosphomannose synthase, β-mannose transferase I, β-mannose transferase II, β-mannose transferase III and β-mannose transferase IV of receptor Pichia pastoris, to obtain recombinant yeast 1;
(A2) expressing at least one of the following exogenous proteins in the recombinant yeast 1: exogenous mannosidase I, exogenous N-acetylglucosamine transferase I, exogenous mannosidase II, exogenous N- acetylglucosamine transferase II, exogenous galactose isomerase and exogenous galactose transferase to obtain recombinant yeast 2; the recombinant yeast 2 is a yeast engineered strain having the ability to modify a specific mammalian cell glycoform;
wherein the specific mammalian cell glycoform is GalₐGlcNAc_{b}Man_{c}GlcNAc₂, wherein a: 0-2; b: 0-2; c: 3-5.

51. The method according to claim 50, wherein the method further comprises the following steps (A3):
(A3) inactivating the endogenous O-mannose transferase I of the recombinant yeast 2 to obtain recombinant yeast 3; wherein the recombinant yeast 3 is also a yeast engineered strain having the ability to modify a specific mammalian cell glycoform.

52. The method according to claim 50, wherein when the specific mammalian cell glycoform is Man₅GlcNAc₂, the exogenous protein expressed in the recombinant yeast 1 in step (A2) is exogenous mannosidase I;
when the specific mammalian cell glycoform is GlcNAcMan₅GlcNAc₂, the exogenous protein expressed in the recombinant yeast 1 in step (A2) is exogenous mannosidase I and exogenous N-acetylglucosamine transferase I;
when the specific mammalian cell glycoform is GalGlcNAcMan₅GlcNAc₂, the exogenous protein expressed in the recombinant yeast 1 in step (A2) is exogenous mannosidase I, exogenous N-acetylglucosamine transferase I, exogenous galactose isomerase and exogenous galactose transferase;
when the specific mammalian cell glycoform is GalGlcNAcMan₃GlcNAc₂, the exogenous protein expressed in the recombinant yeast 1 in step (A2) is exogenous mannosidase I, exogenous N-acetylglucosamine transferase I, exogenous galactose isomerase exogenous galactose transferase, and exogenous mannosidase II;
when the specific mammalian cell glycoform is Gal₂GlcNAc₂Man₃GlcNAc₂, the exogenous protein expressed in the recombinant yeast 1 in step (A2) is exogenous mannosidase I, exogenous N-acetylglucosamine transferase I, exogenous galactose isomerase and exogenous galactose transferase, exogenous mannosidase II, and exogenous N-acetylglucosamine transferase II.

53. The method according to claim 50, wherein that in step (A1), the inactivating endogenous α-1,6-mannose transferase, phosphomannose transferase, phosphomannose synthetase, β-mannose transferase I, β-mannose transferase II, β-mannose transferase III and β-mannose transferase IV of the receptor Pichia pastoris are all knocked out by homologous recombination;
alternatively, in step (A2), expressing the exogenous protein in the recombinant yeast 1 is achieved by introducing a gene encoding the exogenous protein into the recombinant yeast 1;
alternatively, in step (A2), the exogenous mannosidase I is expressed and localized in the endoplasmic reticulum;
alternatively, in step (A2), the exogenous N-acetylglucosamine transferase I is expressed and localized in the endoplasmic reticulum or medial Golgi apparatus;
alternatively, in step (A2), the exogenous mannosidase II is expressed and localized in the endoplasmic reticulum or medial Golgi apparatus;
alternatively, in step (A2), the exogenous N-acetylglucosamine transferase II is expressed and localized in the endoplasmic reticulum or medial Golgi apparatus;
alternatively, in step (A2), the exogenous galactose isomerase and the exogenous galactose transferase are expressed and localized in the endoplasmic reticulum or medial Golgi apparatus; and
alternatively, in step (A3), inactivating the endogenous O-mannose transferase I of the recombinant yeast 2 is achieved by inserting and inactivating the gene encoding O-mannose transferase I in the genomic DNA of the recombinant yeast 2.

54. The method according to claim 53, wherein the gene encoding the exogenous protein is introduced into the recombinant yeast 1 in the form of a recombinant vector.

55. The method according to claim 53, wherein both the gene encoding exogenous mannosidase I and the gene encoding exogenous mannosidase II are introduced into the recombinant yeast 1 twice.

56. The method according to claim 53, wherein the exogenous mannosidase I is derived from trichoderma viride, and is fused with the endoplasmic reticulum retention signal HDEL at the C-terminus.

57. The method according to claim 53, wherein the exogenous N-acetylglucosamine transferase I is derived from mammals, and is fused with an endoplasmic reticulum or medial Golgi apparatus localization signal at the N-terminal or C-terminal.

58. The method according to claim 57, wherein the exogenous N-acetylglucosamine transferase I is derived from humans and contains mnn9 localization signal.

59. The method according to claim 53, wherein the exogenous mannosidase II is derived from filamentous fungi, plants, insects, Java or mammals, and is fused with an endoplasmic reticulum or medial Golgi apparatus localization signal at the N-terminal or C-terminal.

60. The method according to claim 53, wherein the exogenous N-acetylglucosamine transferase II is derived from mammals, and is fused with an endoplasmic reticulum or medial Golgi apparatus localization signal at the N-terminal or C-terminal.

61. The method according to claim 60, wherein the exogenous N-acetylglucosamine transferase II is derived from humans, and both contain mnn2 localization signal.

62. The method according to claim 50, wherein the exogenous mannosidase II is derived from nematodes and contains mnn2 localization signal.

63. The method according to claim 53, wherein both the exogenous galactose isomerase and the exogenous galactose transferase are derived from mammals, and are fused with an endoplasmic reticulum or medial Golgi apparatus localization signal at the N-terminal or C-terminal.

64. The method according to claim 63, wherein the exogenous galactose isomerase and the exogenous galactose transferase are fusion proteins, both of which are derived from humans, and share a kre2 localization signal.

65. The method according to claim 50, wherein the α-1,6-mannose transferase is the following B1) or B2):
B1) a protein whose amino acid sequence is SEQ ID No.1;
B2) a protein having the same function as the amino acid sequence shown in SEQ ID No.1 through substitution and/or deletion and/or addition of one or several amino acid residues, or a protein having more than 99%, more than 95%, more than 90%, more than 85% or more than 80% homology with the amino acid sequence shown in SEQ ID No.1 and having the same function;
alternatively, the phosphomannose transferase is the following B3) or B4):
B3) a protein whose amino acid sequence is SEQ ID No.2;
B4) a protein having the same function as the amino acid sequence shown in SEQ ID No.2 through substitution and/or deletion and/or addition of one or several amino acid residues, or a protein having more than 99%, more than 95%, more than 90%, more than 85% or more than 80% homology with the amino acid sequence shown in SEQ ID No.2 and having the same function;
alternatively, the phosphomannose synthase is the following B5) or B6):
B5) a protein whose amino acid sequence is SEQ ID No.3;
B6) a protein having the same function as the amino acid sequence shown in SEQ ID No.3 through substitution and/or deletion and/or addition of one or several amino acid residues, or a protein having more than 99%, more than 95%, more than 90%, more than 85% or more than 80% homology with the amino acid sequence shown in SEQ ID No.3 and having the same function;
alternatively, the β-mannose transferase I is the following B7) or B8):
B7) a protein whose amino acid sequence is SEQ ID No.4;
B8) a protein having the same function as the amino acid sequence shown in SEQ ID No.4 through substitution and/or deletion and/or addition of one or several amino acid residues, or a protein having more than 99%, more than 95%, more than 90%, more than 85% or more than 80% homology with the amino acid sequence shown in SEQ ID No.4 and having the same function;
alternatively, the β-mannose transferase II is the following B9) or B10):
B9) a protein whose amino acid sequence is SEQ ID No.5;
B10) a protein having the same function as the amino acid sequence shown in SEQ ID No.5 through substitution and/or deletion and/or addition of one or several amino acid residues, or a protein having more than 99%, more than 95%, more than 90%, more than 85% or more than 80% homology with the amino acid sequence shown in SEQ ID No.5 and having the same function;
alternatively, the β-mannose transferase III is the following B11) or B12):
B11) a protein whose amino acid sequence is SEQ ID No.6;
B12) a protein having the same function as the amino acid sequence shown in SEQ ID No.6 through substitution and/or deletion and/or addition of one or several amino acid residues, or a protein having more than 99%, more than 95%, more than 90%, more than 85% or more than 80% homology with the amino acid sequence shown in SEQ ID No.6 and having the same function;
alternatively, the β-mannose transferase IV is the following B13) or B14):
B13) a protein whose amino acid sequence is SEQ ID No.7;
B14) a protein having the same function as the amino acid sequence shown in SEQ ID No.7 through substitution and/or deletion and/or addition of one or several amino acid residues, or a protein having more than 99%, more than 95%, more than 90%, more than 85% or more than 80% homology with the amino acid sequence shown in SEQ ID No.7 and having the same function;
alternatively, the O-mannose transferase I is the following B15) or B16):
B15) a protein whose amino acid sequence is SEQ ID No.8;
B16) a protein having the same function as the amino acid sequence shown in SEQ ID No.8 through substitution and/or deletion and/or addition of one or several amino acid residues, or a protein having more than 99%, more than 95%, more than 90%, more than 85% or more than 80% homology with the amino acid sequence shown in SEQ ID No.8 and having the same function;
alternatively, the exogenous mannosidase I is the following B17) or B18):
B17) a protein whose amino acid sequence is SEQ ID No.9;
B18) a protein having the same function as the amino acid sequence shown in SEQ ID No.9 through substitution and/or deletion and/or addition of one or several amino acid residues, or a protein having more than 99%, more than 95%, more than 90%, more than 85% or more than 80% homology with the amino acid sequence shown in SEQ ID No.9 and having the same function;
alternatively, the exogenous N-acetylglucosamine transferase I is the following B19) or B20):
B19) a protein whose amino acid sequence is SEQ ID No. 10;
B20) a protein having the same function as the amino acid sequence shown in SEQ ID No. 10 through substitution and/or deletion and/or addition of one or several amino acid residues, or a protein having more than 99%, more than 95%, more than 90%, more than 85% or more than 80% homology with the amino acid sequence shown in SEQ ID No. 10 and having the same function;
alternatively, the fusion protein consisting of the galactose isomerase and the galactose transferase is the following B21) or B22):
B21) a protein whose amino acid sequence is SEQ ID No. 11;
B22) a protein having the same function as the amino acid sequence shown in SEQ ID No. 11 through substitution and/or deletion and/or addition of one or several amino acid residues, or a protein having more than 99%, more than 95%, more than 90%, more than 85% or more than 80% homology with the amino acid sequence shown in SEQ ID No. 11 and having the same function;
alternatively, the mannosidase II is the following B23) or B24):
B23) a protein whose amino acid sequence is SEQ ID No. 12;
B24) a protein having the same function as the amino acid sequence shown in SEQ ID No. 12 through substitution and/or deletion and/or addition of one or several amino acid residues, or a protein having more than 99%, more than 95%, more than 90%, more than 85% or more than 80% homology with the amino acid sequence shown in SEQ ID No. 12 and having the same function;
alternatively, the acetylglucosamine transferase II is the following B25) or B26):
B25) a protein whose amino acid sequence is SEQ ID No. 13;
B26) a protein having the same function as the amino acid sequence shown in SEQ ID No. 13 through substitution and/or deletion and/or addition of one or several amino acid residues, or a protein having more than 99%, more than 95%, more than 90%, more than 85% or more than 80% homology with the amino acid sequence shown in SEQ ID No. 13 and having the same function.

66. The method according to claim 50, wherein the gene encoding the exogenous mannosidase I is the following C1) or C2):
C1) a DNA molecule whose amino acid sequence is SEQ ID No. 14;
C2) a DNA molecule having more than 99%, more than 95%, more than 90%, more than 85% or more than 80% homology with the nucleotide sequence shown in SEQ ID No. 14 and encoding the exogenous mannosidase I, or a DNA molecule hybridizing to the DNA molecule defined by C1) under stringent conditions and encoding the exogenous mannosidase I;
alternatively, the gene encoding the exogenous N-acetylglucosamine transferase I is the following C3) or C4):
C3) a DNA molecule whose nucleotide sequence is SEQ ID No. 15;
C4) a DNA molecule having more than 99%, more than 95%, more than 90%, more than 85% or more than 80% homology with the nucleotide sequence shown in SEQ ID No. 15 and encoding the exogenous mannosidase I, or a DNA molecule hybridizing to the DNA molecule defined by C3) under stringent conditions and encoding the N-acetylglucosamine transferase I;
alternatively, the gene encoding the fusion protein consisting of the galactose isomerase and the galactose transferase is the following C5) or C6):
C5) a DNA molecule whose nucleotide sequence is SEQ ID No. 16;
C6) a DNA molecule having more than 99%, more than 95%, more than 90%, more than 85% or more than 80% homology with the nucleotide sequence shown in SEQ ID No. 16 and encoding the exogenous mannosidase I, or a DNA molecule hybridizing to the DNA molecule defined by C5) under stringent conditions and encoding the fusion protein;
alternatively, the gene encoding the mannosidase II is the following C7) or C8):
C7) a DNA molecule whose nucleotide sequence is SEQ ID No.17;
C8) a DNA molecule having more than 99%, more than 95%, more than 90%, more than 85% or more than 80% homology with the nucleotide sequence shown in SEQ ID No.17 and encoding the exogenous mannosidase I, or a DNA molecule hybridizing to the DNA molecule defined by C7) under stringent conditions and encoding the mannosidase II;
alternatively, the gene encoding the N-acetylglucosamine transferase II is the following C9) or C10):
C9) a DNA molecule whose nucleotide sequence is SEQ ID No.18;
C10) a DNA molecule having more than 99%, more than 95%, more than 90%, more than 85% or more than 80% homology with the nucleotide sequence shown in SEQ ID No.18 and encoding the exogenous mannosidase I, or a DNA molecule hybridizing to the DNA molecule defined by C9) under stringent conditions and encoding the N-acetylglucosamine transferase II.

67. A Pichia pastoris engineered strain constructed by the method of claim 50.

68. The Pichia pastoris engineered strain according to claim 67, wherein the Pichia pastoris engineered strain is a strain with a preservation number of CGMCCNo19488 preserved in the China General Microbiological Culture Collection Center.

69. A method for preparing a target protein modified with the specific mammalian cell glycoform, comprising the following steps: introducing an encoding gene capable of encoding the target protein into the Pichia pastoris engineered strain as claimed in claim 67 to obtain a recombinant yeast engineered strain; cultivating the recombinant yeast engineered strain to prepare the target protein with the specific specific mammalian cell glycoform.
